(19) United States
(12) Patent Application Publication
(10) Pub. No.: US 2014/0277292 A1
(43) Pub. Date: Sep. 18, 2014

(54) SYSTEMS AND METHODS FOR LIGHT MODULATION OF AUTONOMIC TONE OR FUNCTION

(71) Applicant: PhotoKinetics, Inc., Salt Lake City, UT (US)

(72) Inventor: Douglas James Steel, Salt Lake City, UT (US)

(73) Assignee: PhotoKinetics, Inc., Salt Lake City, UT (US)

(21) Appl. No.: 14/205,404

(22) Filed: Mar. 12, 2014

Related U.S. Application Data
(60) Provisional application No. 61/793,230, filed on Mar. 15, 2013.

(51) Int. Cl. A61N 5/06

(52) U.S. Cl. CPC A61N 5/0622
USPC 607/88

(57) ABSTRACT

The systems and methods disclosed herein concern the modulation of autonomic function and/or biological processes. The systems and methods are useful for treating or preventing diseases, disorders, or conditions having an autonomic tone or function component. Certain embodiments relate to a system that receives signals from a biological entity to modulate the biological processes of that entity through the modulation of light or other relevant parameters. Alternative embodiments involve the use of sensors to obtain data regarding an individual and using that data to specify a lighting environment. Further embodiments involve systems that collect and transmit sensor data to a controller that designates a selected lighting environment that is useful for modulating, treating or preventing a physiological or pathological state. Additional embodiments utilize a controller that transmits a signal to a driver that specifies a particular lighting environment for an individual in need of modulation, treatment or prevention.

### PRIORITY CLAIM

This application claims the benefit of U.S. Provisional Patent Application Ser. No. 61/793,230 filed Mar. 15, 2013.

### FIELD OF THE INVENTION

The invention is generally in the field of systems and methods for modulating autonomic tone (or function). More specifically, the invention relates to a system that receives signals from a biological entity to modulate the biological processes of that entity through the modulation of light or other relevant parameters.

### BACKGROUND OF THE INVENTION

The eye contains a population of rod cells for sensing intensity of light and three populations of cone cells for sensing color. A series of recent findings have shown that the eye possesses an additional population of photosensitive cells located in the retinal ganglion cellular layer known as Intrinsically-Photosensitive Retinal Ganglion Cells (ipRGCs) or Melanopsin Cells, which mediate non-visual responses to light. These cells are responsive only to wavelengths of light in a range of approximately 440-520 nm. In addition, these cells form connections to a pathway projecting from the front of the retina to the Suprachiasmatic Nucleus and proximal Hypothalamic regions (including the Lateral and Anterior Nuclei, and the Sub-Paraventricular Zone), the Olivary Pretectal Nucleus, Intergeniculate Leaf, and Dorso-lateral and Ventro-lateral Geniculate Nuclei of the Thalamus, and a projection pathway to the Medulla in the hindbrain. This network appears not be used in vision, but in other physiological processes including autonomic function.

The autonomic nervous system (primarily composed of the sympathetic and parasympathetic systems, governs adaptation to changing environments such as physical threats or changes in temperature. The balance between sympathetic and parasympathetic system (referred to as autonomic tone) is implicated either causally or directly in numerous diseases and disorders.

Current methods of treating diseases and disorders characterized by imbalances in autonomic tone (or function) involve pharmaceutical treatments or behavioral changes (i.e., therapy). Therefore, there is a need in the art for methods and systems for modulation of autonomic tone or function. Products capable of affecting the ipRGC response would have the advantage of more effectively treating or preventing a variety of conditions.

### BRIEF SUMMARY OF THE INVENTION

A system and method for modulating autonomic function or biological processes is provided herein. The system and method is useful for treating diseases, disorders, or conditions having an autonomic tone or function component. The system and method is also useful for preventing diseases, disorders, or conditions having an autonomic tone component. The system involves the use of sensors to obtain data regarding an individual, such as a human, in need of such treatment or prevention, and using this data to control or specify a lighting environment. The systems and method can also be used to modulate biological processes in organisms besides humans such as cell cultures, invertebrates, and mammals like rodents, canines, felines and non-human primates. The system and method typical involve one or more sensors that provide measures of physiological or pathological states. The measures of physiological or pathological states obtained from the sensor data are transmitted to and processed by a controller that designates a selected (or desired) lighting environment (and optional additional environmental parameters) that is useful for modulating, treating, or preventing a physiological or pathological state. The controller transmits to a driver a signal that drives or specifies a particular lighting environment for the individual in need of modulation, treatment, or prevention.

Modulation of the lighting environment by the systems and methods described herein involves specifying an amount of light in one or more regions of the visible spectrum that is to be provided to the individual or organism. The one or more regions of the visible spectrum refer to one or more of 700-640 nm (red); 640-625 nm (orange-red); 625-615 nm (orange); 615-600 nm (amber); 600-585 nm (yellow); 585-555 nm (yellow-green); 555-520 nm (green); 520-480 nm (blue-green); 480-450 nm (blue); 450-440 nm (indigo); 440-430 nm (indigo), or 430-395 nm (violet).Modulation of the lighting environment as described herein, in one specific implementation, involves specifying an amount of light in the range of the visible spectrum involved in ipRGC activity. Typically, light in the range of 440 nm to 520 nm that is involved in ipRGC activity is specified.

### BRIEF DESCRIPTION OF THE DRAWINGS

FIG. 1A illustrates a non-limiting example of a system according to an embodiment of the present invention. FIG. 1B illustrates the expected outcome of use of the technology described herein in a hypervigilant study according to an embodiment of the present invention. FIG. 1C illustrates the expected outcome of use of the technology described herein to modulate autonomic tone as expressed as a ratio of parasympathetic activity or tone to sympathetic activity or tone (units are arbitrary e.g., the measures of sympathetic activity and parasympathetic activity are multiplied by a correction or normalization factor; the autonomic tone value of 1 indicates a healthy balance of sympathetic and parasympathetic activity) according to an embodiment of the present invention. FIG. 1D illustrates the expected outcome of use of the technology described herein to modulate sympathetic activity (units are arbitrary e.g., the measures of sympathetic activity) according to an embodiment of the present invention. FIG. 1E illustrates the expected outcome of use of the technology described herein to modulate parasympathetic activity (units are arbitrary e.g., the measures of parasympathetic activity are multiplied by a correction or normalization factor) according to an embodiment of the present invention.

FIG. 2 illustrates another non-limiting example of a system according to an embodiment of the present invention.

FIG. 3 illustrates a non-limiting method according to an embodiment of the present invention.

FIG. 4 illustrates a non-limiting example of a controller according to an embodiment of the present invention.

The Figures illustrate specific aspects of the systems and method for modulating autonomic tone or function. Together with the following description, the Figures demonstrate and explain the principles of the methods and compositions produced through these methods. In the drawings, the thickness of layers and regions are exaggerated for clarity. The same reference numerals in different drawings represent the same element, and thus their descriptions will not be repeated. As the terms “on”, “attached to”, or “coupled to” are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION OF THE INVENTION

The following description supplies specific details in order to provide a thorough understanding of the methods, systems and optical filter products derived therefrom as described herein. Nevertheless, the skilled artisan would understand that the systems and methods of modulating autonomic tone or function can be implemented and used without employing these specific details. Indeed, the systems and methods can be placed into practice by modifying the illustrated systems and methods and can be used in conjunction with any other materials and techniques conventionally used in the industry. For example, while the description refers to specific hardware (and configurations) and diseases (and/or disorders), in particular aspects alternative hardware and hardware configurations could be modified to be used with or to develop other systems for modulating autonomic tone or function or other physiological or pathological parameters.

As disclosed herein, systems and methodology to modulate autonomic tone or function are provided including autonomic tone, sympathetic activity, parasympathetic activity, autonomic reactivity, sympathetic reactivity or parasympathetic reactivity. The systems and methods may be used to treat diseases and condition that are associated with imbalances in autonomic tone (or e.g., autonomic dysfunction) or that may be treated or prevented by modulating autonomic tone or function. The systems and methods described herein involve sensing one or more autonomic tone indices (ATI) in an individual or group of individuals. Alternatively, or simultaneously, the systems and methods can sense one or more disease (or disorder) specific indices (DSI). The ATI or DSI are obtained through the use of sensors that are related to a specific physiological process. The ATI or DSI can be monitored continuously or in a variety of time increments depending on the particular parameter being sensed. For example, heart rate or related indices maybe sensed in a continuous manner using a heart rate monitor. Other indices can be monitored at appropriate intervals and/or times of day e.g., blood levels of various analytes, salivary cortisol, or salivary alpha-amylase using appropriate detection systems. According to the systems and method described herein, these indices (ATI and/or DSI) are used to determine an appropriate modulation of ipRGC activation through control of the lighting that an individual (or group of individuals) is exposed. Thus, the systems and methods described herein further involve lighting (e.g., illumination) or light filters (e.g., optical filters) that can modulate light in the range of ipRGC activation (typically in the range of 440 nm to 520 nm). Additionally, the systems and methods described herein can optionally optimize the color rendering of the illumination or optical filter, the relative transmitted luminance of a filter, luminosity, luminance, the ability to select the performance of the filter or illumination with respect to its ability to better control retinal ganglion (ipRGC) activity, and/or other characteristics of light like color temperature or color balance. The systems and methods can attenuate ipRGC activity or permit a selected ipRGC activity up to full saturation of ipRGC activity. The system and methods described herein can also take into consideration the nature of lighting, referred to as the predominant lighting environment (e.g., sunlight, fluorescent, or incandescent), most commonly experienced by the individual, and can also adjust for the level of lighting. For example, the individual (or individuals) can select the type of lightning environment that they are in or this can be measured by an additional sensor or sensors. This information, in combination with the ATI and/or DSI can be used to determine a selected level of ipRGC modulation that is desirable to affect a physiological or pathological condition, thus identifying or providing a selected or desirable lighting environment. In an alternative configuration, the systems and methods can be used to modulate physiological or other relevant parameters of non-human organism including non-human primates, rodents, felines, canines, invertebrates or e.g., cell culture.

Thus in some embodiments, the systems and methods involve an illuminant or optical filter that produces or filters light in the range of the light spectrum that modulates ipRGC activity. The illuminant or optical filter can optionally produce or modulate light in one or more other regions of the spectrum outside of the ipRGC range to enhance or modulate other light properties for a variety of purposes (e.g., color rendering, adequate illumination, luminance, luminosity, color temperature, color balance, prevention of damage to the eye (e.g., retina) or modulation of ipRGC signal through rod and/or cone mediated pathways). The system advantageously allows for adjustment of spectral profile, lux level and other parameters of the lighting, thereby providing systems that provided for a selected level of ipRGC attenuation or activity based on the nature of light (spectral profile), the lux level, and other parameters (e.g., color rendering) for an optimized lighting environment. In some configurations, the systems and methods described herein may involve determining a desired level of ipRGC activation or attenuation (based on, at least in part, one or more ATI or DSI), determining or estimating the illuminant environment (to take into account the predominant illuminant), and specifying an alteration in the lighting environment to achieve the desired level of ipRGC activation or attenuation (e.g., a desired or selected lighting environment) thereby treating, preventing or modulating a physiological or pathological state. In a simplistic representation, a desired level of ipRGC activation or attenuation is achieved by determining the amount of illumination needed over the relevant part of the visible spectrum (whether it is an increase or decrease) in addition to the level of ipRGC activation provided by the predominant illuminant (or predominant illuminant environment). In some situations, instead of increasing light in the ipRGC range it may be necessary to reduce light in the ipRGC range which may involve the use of optical filters as described herein to reduce the amount of light in the ipRGC range. In this case, the systems and methods may specify the use of one or more optical filters by the individual to accomplish this goal. In addition, systems are provided that correct for scattered light which might not enter directly through the filters (e.g., lenses). For example, an optical filter (e.g., glasses) can have side lighting shielding (similar to that present on common safety glasses) or fit firmly to the face in a wrap-around type product which excludes substantially all light except that which passes through the filter (e.g., lenses) to achieve a desired ipRGC attenuation or activity profile. Other characteristics of light as described herein may be optimized with the use of the systems and methods presented here, such as color rendering, color temperature, color balance, luminance, luminosity, etc.

In one specific aspect, the system and method of the invention is described in reference to FIG. 1. FIG. 1 shows element 10 which represents a subject (e.g., an individual (human), group of individuals (humans), or other non-human organisms (cell cultures human cells, mice, rats, dogs, etc.). Element 20 represents one or more sensors that are configured to obtain data regarding a state of the subject. Element 20 is functionally linked to subject 10 in a manner that allows for the sensor to obtain data regarding a pathological or physiological state of the subject (e.g., ATI or DSI). The nature of the functional linkage depends on the sensor and controller 30 (e.g., direct via a wire or wireless). 30 is a controller which receives a signal from the one or more sensors 20 and provides a signal to a driver 40 either directly or wirelessly. 30 processes information from the one or more sensors to determine an optimal lighting condition and provides this information to the driver 40 which powers the one or more illuminants 55 through a communication link 45. The one or more illuminants 50 provide light in the range that can modulate ipRGC activity (e.g., 440 to 520 nm, or 460 to 500 nm). Optionally, the controller 30 is functionally linked, 55, to a device 60 which provides information about the predominant lighting environment or spectral irradiance that the subject is exposed to.

Without wishing to be bound by theory, it is believed that the technology disclosed herein is remarkably useful at markedly improving outcomes in a variety of diseases especially those having an autonomic component.

In one specific example, the technology is used to treat or prevent a psychiatric (or psychological) disease. A subject may complain of or present symptoms of hypervigilance to a healthcare worker (e.g., physician, psychiatrist, or psychologist). The healthcare worker identifies characteristics such as enhanced state of sensory sensitivity or hyperarousal (e.g., accompanied by an exaggerated intensity of behaviors whose purpose is to detect threats); increased physical and psychological arousal; the subject report sweating, increased heart rate, and shallow and rapid breathing, especially in certain situation or response to certain stimuli. The hypervigilance may be a symptom of or related to another disease or disorder including PTSD and anxiety. Hypervigilance may also be in regards to certain stimuli—e.g., patients having a disease or condition associated with chronic pain may be hypervigilant towards pain or stimuli that induce pain. Another example of stimuli is fear or stimuli that may induce fear or anxiety. The healthcare worker, in this situation, prescribes the use of the technology described herein: one or more sensors, a controller, a driver and one or more illuminants (or optical filters) for treating or preventing hypervigilance. More specifically, the subject uses a system having one or more sensors (e.g., that can be for measuring mood or symptoms of hypervigilance like a questionnaire, an autonomic tone index such as plasma cortisol (or salivary); salivary alpha-amylase; heart function; or galvanic skin response. The one or more sensors can be used for a variety of purposes. For example, the one or more sensors can be used to assess improvement in hypervigilance symptoms or the effect of light on physiological parameters to assess whether the (e.g., during treatment with the light or filter provided to subject or at other periods) desired lighting environment is causing a change in autonomic function. The sensors may be used to sense increased sensitivity to stimuli that may induce hypervigilance. The controller receives signals from the one or more sensors to modulate the one or more illuminants or specify the use of an optical filter (e.g., to provide the desired or selected lighting environment). For example, the controller specifies an effective light doses e.g., in the ipRGC range, which may be a reduction in lighting or specification of the use of an optical filter (e.g., eyewear). During the course of treatment, the data from the one or more sensors are received by the controller and processed internally as a function of the controller (and/or transmitted to a healthcare worker to determine the effectiveness of the treatment or assist in specify the selected or desired lighting environment). The data may indicate that the light treatment is effective; not effective for improving symptoms of hypervigilance; or identify situations where hypervigilance is induced. For example, the hypervigilance questionnaire may indicate improvement in symptoms. The sensors may detect decreased indices like cortisol levels or heart function parameters that indicate a decreased activation of ipRGC. The sensors may indicate an increase in parameters that indicate exacerbation of the hypervigilant condition. The controller may then alter the light dose by altering the illuminant or optical filter (e.g., intensity, timing during day, amount of time per day, etc). Alternatively, the data obtained from a sensor of an autonomic tone index may indicate that during the actual course of light treatment (e.g., when the subject is exposed to light from the one or more illuminants or using eyewear designed to modulate ipRGC activation) the index is not changing in a manner that indicates the light dose is appropriate. The controller may then specify a change in light dose e.g., intensity, timing during day, amount of time per day, etc. For example, the sensor may indicate autonomic elevated indices associated with hypervigilance and the light dose is altered to provide a dose that reduces autonomic hyperactivation and provides symptom improvement in hypervigilance symptoms. Or the sensor information indicates that the light dosage should be altered (e.g., intensity or duration) to achieve the desired improvement in hypervigilance symptoms. According to the technology disclosed herein, the controller transmits a desired lighting environment based to a driver (or the subject) which effectuates the lighting environment. The controller may also transmit this information to another party e.g., healthcare worker that may review the data obtained from the sensors to determine a desired lighting environment. In this case, the information regarding the desired lighting environment may be transmitted to the subject or the controller. FIG. 1B shows that with the use of the technology described herein in hypervigilant subjects, cortisol levels are expected to decrease as compared to subjects not using the technology described herein.

FIG. 1C-1E illustrate contemplated effects of the use of the system and method described herein on measures of autonomic function.

In one specific example, the technology is used to treat or prevent rheumatoid arthritis (RA). A subject may complain of or present symptoms of rheumatoid arthritis to a healthcare worker (e.g., physician, psychiatrist, or psychologist). The healthcare worker, in this situation, prescribes the use of the technology described herein: one or more sensors, a controller, a driver and one or more illuminants (or optical filters) for treating or preventing rheumatoid arthritis. More specifically, the subject uses a system having one or more sensors (e.g., that can be for measuring symptoms of rheumatoid arthritis like a questionnaire (pain), an autonomic tone index such as plasma cortisol (or salivary), or another marker characteristically associated with RA. The one or more sensors can be used for a variety of purposes. For example, the one or more sensors can be used to assess improvement in RA symptoms or the effect of light on physiological parameters (e.g., during treatment with the light provided to subject or at other periods). The controller receives signals from the one or more sensors to modulate the one or more illuminants (e.g., the desired or selected lighting environment) to treat or prevent RA or symptoms thereof. For example, the controller specifies an effective light dose including light in the ipRGC range. During the course of treatment, the data from the one or more sensors are received by the controller and processed internally as a function of the controller or transmitted to a healthcare worker to determine the effectiveness of the treatment. The data may indicate that the light treatment is effective or not effective for improving symptoms of RA. For example, the pain questionnaire may indicate improvement in symptoms or that the symptoms of RA are not improving. The controller may then alter the light dose by altering the illuminant or optical filter e.g., intensity, timing during day, amount of time per day, etc. Alternatively, the data obtained from a sensor of an autonomic tone index may indicate that during the actual course of light treatment (e.g., when the subject is exposed to light from the one or more illuminants or optical filter) the index is not changing in a manner that indicates the light dose is appropriate. The controller may then specify a change in light dose e.g., intensity, timing during day, amount of time per day, etc. For example, the sensor information indicates that the light dosage should be increased (e.g., intensity or duration) to achieve the desired improvement in RA symptoms. According to the technology disclosed herein, the controller transmits a desired lighting environment based on this information to a driver (or individual) which effectuates the lighting environment. The controller may also transmit this information to another party e.g., healthcare worker that may review the data obtained from the sensors to determine a desired lighting environment. In this case, the information regarding the desired lighting environment may be transmitted to the subject or the controller.

In one specific aspect, the system and method of the invention can be described in reference to FIG. 2. FIG. 2 shows element 10 which represents a subject (e.g., an individual (human), group of individuals (humans), or other non-human organisms (cell cultures, human cells, mice, rats, dogs, etc.). Element 20 represents one or more sensors that are configured to obtain data regarding a state of the subject. Element 20 is functionally linked to subject 10 in a manner that allows for the sensor to obtain data regarding a state of the subject. The nature of the functional linkage depends on the sensor. 30 is a controller which receives a signal from the one or more sensors 20 and provides a signal to a driver 40 either directly or wirelessly. 30 process information from the one or more sensors to determine an optimal lighting condition and provides this information to the driver 40 which powers the one or more illuminants 55 through a communication link 45. The one or more illuminants 50 provide light in the range that can modulate ipRGC activity (e.g., 440 to 520 nm, or 460 to 500 nm). Optionally, the controller 30 is functionally linked, 55, to a device 60 which provides information about the predominant lighting environment or spectral irradiance that the subject is exposed to. The driver 40 is functionally linked, 47, to one or more illuminants that provide light not in the ipRGC range (e.g., 700-640 nm (red), 640-625 nm (orange-red), 625-615 nm (orange), 615-600 nm (amber), 600-585 nm (yellow), 585-555 nm (yellow-green), 555-520 nm (green), or 430-395 nm (violet) to modulate ipRGC activity via rods or cones or other properties of the light such as color rendering, color temperature, color balance, luminance, or luminosity.

As used herein, the term “color rendering” refers to the accuracy with which colors are rendered by one illuminant relative to a reference illuminant. Color Rendering Index (CRI) is an indication of how well the illuminant is matched to the reference illuminant, with a CRI≡100 being a perfect match of the illuminant to the reference illuminant. For example, the CRI of filtered sunlight (e.g., sunlight filtered through an optical filter such as a sunglass lens) can be calculated relative to unfiltered sunlight (which would act as the reference illuminant), or can be calculated relative to a theoretical reference illuminant (e.g., Standard D65). CRI relates to color difference such that 4.6 CRI units are about equivalent to DE=1 color difference unit. In this way, just-perceptible changes in CRI occur between the following points: 100, 95.4, 90.8, 86.2, 81.6, and so on (even below zero in some instances). CRI can be determined by calculating color difference between the illuminant and the reference illuminant and applying adaptation models to determine the appropriate perceived CRI. CRI can be determined using CIE 13.3. However, CIE 13.3 is only specified here as an illustrative example of the means by which color rendering can be determined. Other color rendering methods using different color difference and color adaptation procedures can be used such as CRI-109, CRI DE00-109 or any other formulation. In a particular embodiment, the CRI-109 method is defined here in which the color adaptation model of CIE 13.3 is replaced with that from CIE 109.2.

As used herein, the term “Luminance” generally refers to a photometric measure of the luminous intensity per unit area of light traveling in a given direction. As used in this disclosure, “luminance” refers to wavelengths of light in the range that are perceptible to humans (e.g., of a visible sensation to humans) averaged over the visible spectrum of between about 360 nm and about 830 nm, weighted by the photopic function. As such, reduction in luminance of an optical filter may be described as the luminance of light from a reference illuminant filtered by the optical filter relative to the luminance of unfiltered light from the reference illuminant.

“Luminosity” or “luminous intensity” refers to perceived brightness of illumination. Luminosity can, for example, be calculated using (1) the Standard Vision Theory model in which luminosity is determined from luminance (Y), which is itself derived from the Photopic function; (2) the Helmholtz-Kohlrausch model in which luminosity may be determined from luminance (Y) and chromaticity (x,y); and/or (3) the opponent color theory in which luminosity may be determined from L*a*b* coordinates; and/or (4) by empirical brightness determinations. Where reference is made to reducing luminance, it may, additionally or alternatively, include reducing luminosity and/or reducing perceived brightness (theoretical and/or experimental).

A “predominant illuminant environment” refers to the profile of light that a subject is exposed to in a particular setting. For example, the predominant illuminant environment of e.g., a subject in an office setting can comprise sunlight that enters through a window in the office, the fluorescent or incandescent lights the illuminate the office and the light that is emitted from an electronic display (e.g., computer screen) in the office.

A “predominant illuminant” refers to the dominating profile of light that a subject is exposed to in a particular setting. For example, the predominant illuminant is the one that represents 50%, 60%, 70%, 80%, 90% or greater of the luminosity that the individual is exposed to.

As used herein, “desired” or “selected”, “lighting environment” refers to the profile of light that a subject is exposed to as a result of the systems and methods described herein.

As used herein, “operably linked” refers to the functional linkage of two or more components of the systems described herein. Typically operably linkage refers to the ability of two or more of the components to be able to exchange information with one another. This exchange may be unidirectional, bi-directional or multi-directional.

As used herein, the term “optical filter” refers to an ophthalmic product or a coating (e.g., glass or plastic coating) that alters light emitted from an illuminant. Ophthalmic products include e.g., prescription or non-prescription ophthalmic lenses used, for clear or tinted eyeglasses (or spectacles), sunglasses, goggles (e.g., sport or protective), contact lenses with and without visibility tinting or cosmetic tinting. Ophthalmic products can also include thin-film sheets that can be applied to windows or computer monitors for purposes of selectively filtering transmitted light. Ophthalmic products also include devices such as a corneal inlay or onlay that may be configured to filter light. Ophthalmic products for illuminants or window do not necessarily involve a coating but can be configured as part of the product by selecting the appropriate material for altering light.

As used herein, the term “Light” generally refers to radiation in the visible range of the electromagnetic spectrum and parts of that effect vision e.g., UV.

As used herein, the terms “treating”, “preventing”, or “modulating” in the context of a target indication (e.g., disease, disorder or condition) refers to treating, preventing or modulating the target indication or a symptom thereof.

Thus, in one embodiment a system is provided comprising: one or more sensors for measuring one or more physiological or pathological parameters in an individual; a controller for receiving values from the one or more sensor for measuring physiological or pathological parameters in an individual; and one or more illuminants or one or more optical filers configured to modulate autonomic function in an individual through modulation of light in the ipRGC range. In one aspect of this embodiment, the one or more physiological or pathological parameters are one or more ATI. In one aspect, the one or more ATI are heart function, a salivary biomarker, or a blood biomarker. In one aspect of this embodiment, the one or more physiological or pathological parameters are one or more ATI and one or more DSI. In one aspect of this embodiment, the one or more illuminants are one or more illuminants that produce or modulate light in the ipRGC range at levels and durations sufficient to modulate ipRGC activity. In some aspects of this embodiment, the system may involve the use of one or more optical filters. In one aspect of this embodiment, the one or more illuminants are one or more illuminants that produce or modulate light in the range of 440 nm to 520 nm, 450 nm to 510 nm, or 460 nm to 500 nm. In one aspect, the one or more illuminants are one or more LEDs or OLEDs.

In another embodiment, a system is provided comprising: one or more sensors for measuring physiological or pathological parameters in an individual operably linked to a controller; a controller for receiving values from the one or more sensor for measuring physiological or pathological parameters in an individual, a driver for driving the one or more illuminants and one or more illuminants; and one or more illuminants operably linked to said controller configured to modulate autonomic function in an individual by modulation of ipRGC activity. In some aspects of this embodiment, the system may further comprise one or more optical filters.

In yet another embodiment, a system is provided comprising: one or more sensors for measuring one or more physiological or pathological parameters of an individual, operably linked to a controller; a controller for receiving values from the one or more sensor for measuring one or more physiological or physiological parameters in an individual, driver for driving the one or more illuminants operably linked to the controller and one or more illuminants; one or more illuminants operably linked to said controller configured to modulate autonomic function in an individual. In some aspects of this embodiment, the system may further comprise one or more optical filters. In some aspect of this embodiment, the one or more illuminants provide light in the ipRGC range. Thus, the one or more illuminants provide light in the range of 440 nm to 520 nm, 450 nm to 510 nm or 460 nm to 500 nm, where the one or more illuminants are configured to expose an individual to irradiances of at least 10⁴, 10⁵, 10⁶, 10⁷, 10⁸, 10⁹ or 10¹⁰ photons/cm²/sec over a time period sufficient to modulate ipRGC activity. In some aspects, the one or more illuminants for the range of light in the 440 nm to 520 nm, 450 nm to 510 nm or 460 nm to 500 nm range are configured to expose an individual to irradiances 10⁶ to 10¹⁶ photons/cm²/sec over a time period sufficient to modulate ipRGC activity. In some aspects of this system, the one or more optical filters for the range of light in the 440 nm to 520 nm, 450 nm to 510 nm or 460 nm to 500 nm range are configured to expose an individual to irradiances of less than 10⁴, 10⁵, 10⁶, 10⁷, or 10⁸ photons/cm²/sec over a time period sufficient to modulate ipRGC activity. In some aspects, the one or more optical filters for the range of light in the 440 nm to 520 nm, 450 nm to 510 nm or 460 nm to 500 nm range are configured to expose an individual to irradiances of 10⁰ to 10⁸ photons/cm²/sec (or 10¹ to 10⁶ photons/cm²/sec) over a time period sufficient to modulate ipRGC activity. In some aspects of this system, the lighting environment can be additionally be optimized for other lighting characteristic such as luminance, luminosity or color rendering.

In another embodiment, a system is provided comprising: one or more sensors for measuring one or more physiological or pathological parameters in an individual operably linked to a controller; a controller for receiving values from the one or more sensor for measuring physiological or pathological parameters in an individual, a driver for driving the one or more illuminants, operably linked to the controller, and one or more illuminants; and one or more illuminants operably linked to said controller configured to modulate autonomic function in an individual. According to this embodiment, controller uses one or more ATI or DSI to determine a desired or selected lighting environment. In some aspects of this embodiment, the system may further comprise one or more optical filters.

In some aspects of the systems and methods described herein, the subjects living or working lighting environment are measured by a spectral irradiance meter. The spectral irradiance meter (e.g., portable) can be carried by the subject throughout a typical day to measure the lighting environment and then based of this information the optimized filter can be appropriately specified by the optimization process described herein. The spectral irradiance meter can be configured to measure light at any wavelength or range of wavelengths. In a specific aspect, the wavelength or range of wavelengths is within the ipRGC response range. In some aspects, the spectral irradiance meter has a USB interface. For example such data may be recorded on a memory storage device such as a USB flash drive, SD card, mini-SD card, smartphone or similar portable electronic device. One such example of a spectral irradiance meter is the Konica/Minolta SpectraRad (made by B&W Tek Newark, Del.) although other appropriate meters can be used. As described herein below, the spectral irradiance meter or device can be used to determine e.g., the ambient lighting conditions. Another light meter useful for including in the systems and methods described herein is the Daysimeter (or a similar one) developed at the Rensselaer Polytechnic Institute's Lighting Research Center (LRC) in Troy, N.Y. In some aspects, the measurement of light is preferably an estimate of light that is incident to the retina.

In some aspects of these embodiments, the systems and methods can further comprise a device that estimates the spectral irradiance that the individual (or group of individuals) is exposed to.

In some aspects of these embodiments, the systems and methods can further comprise a device that compares the estimate of spectral irradiance that the individual is exposed to with an autonomic tone parameter derived from the one or more sensors for measuring physiological (or pathological) parameters in the individual that provides signal to the controller used to determine light in addition to that of the predominant lighting environment to achieve a selected or desired lighting environment.

In one embodiment, a method of modulating a physiological or pathological state is provided said method comprising: operably linking one or more sensors for measuring one or more physiological or pathological parameters to an individual; providing data from said one or more sensors to a controller; and driving illumination of one or more illuminants by a driver operably linked to the controller, thereby modulating a physiological or pathological state. In one aspect, the method modulates autonomic function. In one aspect, the method modulates autonomic tone. In one aspect, the method increases sympathetic activity. In one aspect, the method increases parasympathetic activity. In one aspect, the method decreases sympathetic activity. In one aspect, the method decreases parasympathetic activity. In one aspect, the method increases sympathetic reactivity. In one aspect, the method increases parasympathetic reactivity. In one aspect, the method decreases sympathetic reactivity. In one aspect, the method decreases parasympathetic reactivity. In some aspects, the method comprises (1) determining an increase or decrease in an ATI or DSI. In some aspects, the method comprises (1) determining an increase or decrease in an ATI or DSI and (2) providing a lighting environment that slows, stops or reverses the increase or decrease in the ATI or DSI.

In another embodiment, a method of modulating a physiological or pathological state is provided said method comprising: measuring one or more ATI or DSI in an individual; providing data corresponding to one or more ATI or DSI from said individual to a controller; determining a desired or selected lightning environment based on the one or more ATI or DSI; and driving illumination of one or more illuminants by a driver to provide the desired or selected lighting environment, thereby modulating a physiological or pathological state. In one aspect, the method increases sympathetic activity. In one aspect, the method modulates autonomic function. In one aspect, the method modulates autonomic tone. In one aspect, the method increases parasympathetic activity. In one aspect, the method decreases sympathetic activity. In one aspect, the method comprises decreases parasympathetic activity. In one aspect, the method increases sympathetic reactivity. In one aspect, the method increases parasympathetic reactivity. In one aspect, the method decreases sympathetic reactivity. In one aspect, the method decreases parasympathetic reactivity. In some aspects, the method comprises (1) determining an increase or decrease in an ATI or DSI. In some aspects, the method comprises (1) determining an increase or decrease in an ATI or DSI and (2) providing a lighting environment that slows, stops or reverses the increase or decrease in the ATI or DSI.

Indices of autonomic tone or disease specific indices are monitored or modulated in various aspects of the systems and methods provided herein. Typically one or more indices of autonomic tone are measured by a sensor and used to determine a desired lighting environment thereby treating, preventing or modulating one or more diseases, disorders or conditions. The desired lighting environment is selected or determined to be useful in modulating one or more indices or autonomic tone. The sensed indices and modulated indices of autonomic tone may be the same or different indices. Desirably, the modulation of autonomic function brings about a beneficial or desired changed in a physiological or pathological state in an individual. One index of autonomic tone can be used or measured or a plurality of indices of autonomic tone can be used or measured to yield a measured autonomic tone value. In some aspects, the ATI are one or more indices of sympathetic activity and one or more indices of parasympathetic activity. The measured autonomic tone indices are compared to a desired autonomic tone value and a desired or selected lighting environment is determined, at least in part, based on a comparison of the measured autonomic tone indices and the desired autonomic tone value. The desired lighting environment can also be determined, in part, by the desired change in physiological or pathological state.

The one or more autonomic tone indices (ATI) that can be used in the systems and methods described herein can be selected from any parameters that provided a measure of autonomic tone. The systems and methods provided herein can include sensors for (or measuring) one or more ATI, 2 ATI, 3 ATI, 4 ATI, 5 ATI, 6 ATI, 7 ATI, 8 ATI, 9 ATI, or 10 or more ATI and/or one or more DSI, 2 DSI, 3 DSI, 4 DSI, 5 DSI, 6 DSI, 7 DSI, 8 DSI, 9 DSI, or 10 or more DSI. In some aspects, autonomic tone is estimated by measuring peripheral autonomic tone. In some aspects, autonomic tone is estimated by measuring central autonomic tone. For examples the one or more ATI is selected from a heart function parameter, galvanic skin response, a salivary biomarker or a blood biomarker. In another aspect, the ATI is. In another aspect, the ATI is heart rate, heart rate variability, the LF component of heart rate variability, the RF component of heart rate variability, melatonin levels, cortisol levels (blood or salivary), or galvanic skin response. In yet another aspect, the ATI is (1) heart function (e.g., blood pressure, flow or pulse), (2) gastrointestinal activity (e.g., movements and secretions), (3) body temperature, (4) bronchial dilation, (5) blood glucose levels, (6) metabolism, (7) micturition and defecation, (8) pupillary light and accommodation reflexes, (9) glandular secretions, (10) heart rate variability (e.g., RF or LF component). In some aspects, the ATI is a plasma catecholamine (e.g., dopamine, epinephrine, and norepinephrine), a urinary catecholamine, or urinary metanephrines. In some aspects, the ATI is adrenocorticotropic hormone. In some aspects, the ATI is the LF component of HRV. The LF component of HRV represents both sympathetic and parasympathetic nerve activity. In one aspect, the ATI is the HF component of HRV. The HF component represents almost entirely parasympathetic nerve activity. In one aspect, the ATI is the LF/HF ratio which represents relative sympathetic nerve activity. Non-limiting examples of sensors for sensing ATI and DSI are disclosed herein, including the generic and specific examples disclosed in the Examples.

One measure of autonomic tone for use in the systems and methods described herein is heart function. One specific measure of heart function is heart rate. Heart rate can be measured by measuring pulse—thus heart rate (or pulse) monitors can be used to measure pulse. Pulse can be measured by any suitable device including, pulse oximetry devices, sphygmomanometer. Another specific measure of heart function is heart rate variability (HRV). HRV can be measured by electrocardiogram (ECG). ECG can also be used to measure heart rate. LF-HRV and HF-HRV. It is contemplated that other measures of heart function can be used as autonomic tone indices. Examples of ECG devices include, but are not limited to, the InstantCheck Handheld ECG-EKG Monitor FP-ICH, the ReadMyHeart Handheld ECG-EKG Monitor FP-RMH, or the Easy ECG Handheld Monitor FP180. In some aspects, the sensor is integrated with another device. For example, iHealth Labs has a pulse monitor (via the sphygmomanometer technique) that integrates with an iPhone type device. Other examples of sensors that may be used in the system and method described herein are the Amiigo fitness sensor or the Nike Fuel and/or associated apps. Sensors may transmit the sensed values to a device which then transmit information to a controller or the value may be transmitted directly to the controller.

Certain molecular markers can be used in the systems and methods described herein as ATI or DSI. Non-limiting examples include salivary cortisol, salivary alpha-amylase, blood melatonin, c-reactive protein (CRP), cytokine or cytokine profiles (e.g., IL-1, IL-6, TNF-alpha, interferon-alpha, interferon-gamma). See e.g., the Examples.

The sensors for use in the systems and methods described herein can be any sensor that measures or estimates one or more indices of autonomic tone (or function) or one or more DSI.

The system(s) and method (s) described herein involve a driver and controller. The controller receives one or more input signals and delivers to the driver one or more output signals for modulation/control of illuminants based on the desired lighting environment determined by the controller function. The controller function can be one device or it can be in more than one device. For example, the a desired lighting environment can be determined in controller “1” which transmits this information to controller “2” which determines how to achieve this desired lighting environment given the characteristics of the one or more illuminant and e.g., the predominant lighting environment, which in turn communicates this information in a form appropriate to the driver (or drivers). The driver (or drivers) provides the appropriate power to the one or more illuminants. Typically, the input signals to the controller are one or more indices of autonomic tone or one or more disease specific indices. The input signal can be an individual index of autonomic function (e.g., a value corresponding to one measured index) or it can be composite value of two or more measured values of autonomic function. The input signal can be processed prior to input into the controller or it can be processed as part of the function of the controller. For example, the composite autonomic tone value can be determined by obtaining values from the one or more sensors and deriving a composite autonomic tone value by averaging the indices of autonomic tone or by weighting each individual autonomic tone value with a weighting factor and determining a composite autonomic tone value through combination of the individual weighted indices of autonomic tone. Thus, the autonomic tone value can arrive as a signal to the controller/driver as a discrete value or the discrete value can be determined within the functionality of the controller by combining values corresponding to the one or more indices of autonomic tone. Based on the autonomic tone value a selected or desired lighting environment is determined. The desired or selected lighting environment determination may also take into account 1, 2, 3 or more of the following: the predominant lighting environment, the predominant illuminant, optimized color rendering, optimized luminosity, optimized luminance, one or more DSI, or the disease, disorder or condition that is to be treated, prevented or modulated.

The controller in the systems and methods described herein function to specify the desired or selected lighting environment. Typically, the controller specifies the lighting environment by communicating to one or more drivers. The driver(s) serve to provide electrical energy to the illuminant or may specify an appropriate optical filter. The controller functions to specify an increase, decrease or maintenance of the intensity of light at a particular wavelength or wavelengths of light based on information obtained from the one or more sensors. In one aspect, the controller can specify an increase in the level of light in the range of 440 to 520 nanometers (nm), decrease the light in the range of 440 to 520 nanometers, or maintain the level of light in the range of 440 to 520 nanometers. In another aspect, the controller can specify an increase in the level of light in the range of 460 to 500 nanometers, decrease the light in the range of 460 to 500 nanometers, or maintain the level of light in the range of 460 to 500 nanometers. An increase, decrease or maintenance of a level of light can refer to the level of light across the entire range, a bandwidth within that range (e.g., 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 22 nm, 24 nm, 26 nm, 28 nm, 30 nm, 33 nm, 36 nm, 39 nm, 42 nm, 46 nm, 50 nm, 55 nm, 60 nm, 65 nm, 70 nm, or 75 nm), or a monochromatic value within that range. Alternatively, the controller can specify that a particular illuminant in the range of light is increased, decreased or maintained (e.g., increase, maintain or decrease the LEDs serving a particular range of light like that in the blue range or green range).

In some aspects, the autonomic tone value or composite value is transmitted to a device (e.g., controller) that receives the value or values and uses this information to transmit another signal to an illuminant driver. The controller can process the received ATI or composite ATI to specify or transmit to the driver a desired or selected lighting environment. The controller may optionally receive information about the predominant illuminant environment and use this information in deriving a desired lightning environment. Furthermore, the controller can use or receive information about a physiological or pathological state of the individual (e.g., a disease or disorder to be treated or prevented). Alternatively, the controller can receive information from another device that receives the ATI or DSI. This information is then processed by the controller which specifies the desired lightning environment.

Typically, the controller has a computer readable medium having computer readable code embodied thereon (e.g., for determining desired or selected lighting environments and/or specifying desired or selected lighting environments to a driver or used) and hardware for integrating with the system described herein e.g., CPU or processor, memory communication ports or means (e.g., WiFi or Bluetooth or other wireless methods).

The controller can be any controller that is capable of receiving one or more input signals corresponding to the one or more ATI or one or more DSI and transmitting one or more output signals to a driver or other device. Data from the one or more sensors is transmitted wirelessly to the controller. Alternatively, the data may be transmitted via a direct connection e.g., a wire. The controller is operably linked to the one or more sensors such that communication of information from the sensor to the controller is possible. In some aspects, the operably linkage is wireless. Examples of controllers or controller systems include, but are not limited to, DMX or Ethernet based controllers. Examples of specific controllers or controller systems that may be integrated with the systems and methods described herein include, but are not limited to, controllers for ColorKinetics from Phillips, Light Systems Manager from Phillips, ColorDial Pro, iColorPad from Phillips, Optronic from Osram, or Ledotron from Osram. The use of commercial available controllers like these may require adaptation of the software or use of a program that derives the selected lighting environment and then specifies this information to the commercially available controller.

Typically, controllers as described herein involve the use of software or applications for specifying lighting environments.

The controller may be integrated with the systems and methods described herein to control the lighting environment providing direct control or remote-access control. One non-limiting example of a controller system is a smartphone based system. Controllers may comprise the use of systems of remote access via an app or cloud-based service or any other suitable interface.

Thus, in one embodiment, the controller determines (1) the spectrum of light delivered; (2) the intensity of light being delivered at one or more specified wavelengths in the visible range; (3) the duration of light exposure; (4) the interval between periods of exposure; (5) the timing of light exposure (e.g., at what points during a 24 hour period or wake cycle); (6) the history of prior light exposure by that person (e.g., have they been living in a bright light or dim light environment); (7) the ambient light conditions in the environment in which the systems and methods are being used; (8) coordinated use of the lighting with the controller to account for the total amount of light in the environment; (9) the functional coupling in the nervous system of the user; and (10) the way in which specific body physiological parameters (ATI or DSI) can be used predictively to “prescribe” a course of light therapy, using biosensors connected to the controller.

Thus, in one embodiment, the controller receives input signals corresponding to 1, 2 or 3 or more of the following (a) history of prior light exposure by the individual; (b) the ambient light conditions in the environment in which the system and method described herein are being used; (c) one or more ATI or DSI; (D) diseases, disorder or condition to be treated (or prevented or modulated); to determine one or more output signals.

Thus, in one embodiment, the controller delivers output signals corresponding to 1, 2, or 3 or more of the following (1) the spectrum of light delivered; (2) the intensity of light being delivered at one or more specified wavelengths; (3) the duration of light exposure; (4) The interval between periods of exposure; (5) the timing of light exposure, based on one or more input signals; (6) or length of light treatment regimen.

For example, the controller may receive information from the one or more input signals and output one or more output signals as follows (see also FIG. 4 where the elements listed below correspond to the indicated number system).

Input Signal(s) to the Controller

History of Light Exposure (HLE) (100): HLE information can be inputted into the controller in a variety of ways and used in variety of manners to aid in determining the output signals of the controller depending on the particular use (e.g., the target disease, condition, or disorder). HLE can be a short term (e.g., within the last 1 minutes, 5 minutes, 10 minutes, 20 minutes, 30 minutes, hour, 2 hours, 4 hours, 6 hours, 12 hours or 24 hours), medium term (within the last day, 2 days, 3 days, 4 days, 5 days, 6 days, 7 days, 2 weeks, 3 weeks, 4 weeks) or long term history (e.g., within the last month, 2 months, 3 months, 4 months, 5 months, 6 months, 9 months, 1 year, 2 years, 3 years, 5 years, 10 years, or 15 years or more). The HLE can range from general to specific information. For example, the HLE may include information about the waking and sleeping hours of the individual, sunrise or sunset times, latitudes, exposure to sunlight during the day, computer or TV or other electronic display exposure during the day or evening, light usage and type of lighting used after dark (e.g., at work or at home), hours and type of light subject is exposed to at home or work, or any other appropriate HLE information. The HLE can be general in the sense of what type of lighting—incandescent, LED, sunlight, etc., or can be detailed with measurements or estimates of spectral irradiance. The HLE can be used in a variety of manners in the context of the systems and methods described herein. For example, it can be used to provide an index or estimate of the effect of light on the target disease, disorder or condition. One non-limiting example is e.g., how a short, medium, or long term HLE has impacted autonomic tone. A short HLE may be used for example in a Jet Lag situation where short term alterations in the lighting experienced by travels moving from time may be useful to incorporate medium or long term HLEs to further characterize this short term process in relation to the long term history. This information can be used in an algorithm of the controller that takes into account other input parameters to specify the one or more output parameters. In another example, medium or long term HLE may be used in a controller algorithm with other input parameters to specify one or more output signals in relation to e.g., medium or long term disruptions in circadian processes, the effect of medium or long term HLE on various diseases like cancer, neurodegeneration, immune system dysfunction, cardiovascular disorders, psychiatric or psychological disorders like PTSD, SAD, depression. Thus, the HLE information may be used in determining a “lighting correction” by specifying one or more output signals based, in part, on this information. Furthermore, the HLE may be used to generate a sensitivity parameter that indicates how sensitive the ipRGC system of the individual is to activation based on HLE.

Ambient Light Conditions (ALC) (102). The ALC is a specific or general measure or estimate of the current lighting conditions that an individual or subject is exposed to. For example, input signal to the controller may be a simple indication of sunlight, darkness, incandescent light, or any other type of light that is selected or specified by the e.g., the user or individual, such an ALC may be specified by selecting the ACL from a list of examples. In some situations, the ACL may be more detailed e.g., actual measurements or characterizations of the light by any number of devices including a spectral irradiance meter. This information can be used in generating one or more output signals with the target disease, disorder or condition taken into account. The ALC can be used to determine the desired or selected lighting environment. The target disease, disorder or condition may be treated (or prevented or modulated) by providing modulation (e.g., attenuation or activation) of ipRGC activity and the ACL is useful for estimating or determining current ipRGC activation based on the ACL (or predominant lighting environment).

Autonomic Tone Indices (ATI) (104): ATI is an input to the controller can be used in several ways in the systems and methods described herein. The ATI input signal provides measures of the current status of autonomic function in the subject or individual and may indicate that modulation (attenuation or activation) of ipRGC activity is desirable for a particular target disease, disorder or condition. In another aspect, the ATI can be used to measure the effect of different lighting environments on the ATI and serve to further refine the lighting environment to achieve the desired results. For example, the systems and methods described herein may result in or provide a new lighting environment for the individual. As the lighting environment is change, it is expected that changes in the ATI or DSI may occur. The changes may be desirable or may be undesirable due to a number of variables in the individual and environment. These changes in the changed be used in the systems and methods to further alter the lighting environment to bring about a desirable change in the ATI or DSI thereby affecting the target disease, disorder or condition e.g., through modulation of autonomic function or tone.

Disease Specific Indices (DSI) (106): DSI is an input to the controller can be used in several ways in the systems and methods described herein. The DSI input signal provides measures of the current status of target disease, condition, or disorder parameters in the subject or individual and may indicate that modulation (attenuation or activation) of ipRGC activity is desirable for a particular target disease, disorder or condition. In another aspect, the DSI can be used to measure the effect of different lighting environments on the DSI and serve to further refine the lighting environment to achieve the desired results. For example, the systems and methods described herein may result in or provide a new lighting environment for the individual. As the lighting environment is change, it is expected that changes in the ATI or DSI may occur. The changes may be desirable or may be undesirable due to a number of variables in the individual and environment. These changes in the changed be used in the systems and methods to further alter the lighting environment to bring about a desirable change in the ATI or DSI thereby affecting the target disease, disorder or condition e.g., through modulation of autonomic function or tone.

Disease, Disorder or Condition (DDC) (108): DDC information is an input to the controller that is used to determine a selected or desired lighting environment that is useful for treatment, prevention or modulation. The selected or desired lighting environment is determined, at least in part, based on the DDC and zero, one or more, other input signals, through the specification of one or more output signals. DDC provides information about the disease, disorder, or condition that system and method are being used by the individual to treat (or prevent or modulate). For example, if the DDC is disruption of circadian function, outputs signals would be generated in the specific context of this condition. In another example, if the DDC is physiological cues indicating the onset of light-induced migraine headache outputs signals would be generated in the specific context of this condition. In another example, if the DDC is physiological cues indicating the onset of epileptic seizure, outputs signals would be generated in the specific context of this condition. In another example, if the DDC is a pattern of change in heart rate variability indicative of the onset of an episodic or chronic cardio-vascular condition or pathology outputs signals would be generated in the specific context of this condition. In another example, if the DDC is a pattern of change in brain wave EEG pattern or power spectrum alteration indicative of the onset of an episodic or chronic neurological or psychological condition or pathology outputs signals would be generated in the specific context of this condition. Such output signals may be in part determined by an appropriate person e.g., physician, nurse, healthcare worker or other individual. Thus, in some aspects, the DDC information may be received as an input, either directly or indirectly, e.g., from a physician, psychiatrist, psychologist, health care worker, supervisor or other individual, or the DDC information may be received as an input signal from the user.

Output Signal(s) from the Controller

Spectrum of Light Delivered (SLD) (120): The SLD can be one of the one or more of the output signals derived from the controller based on the one or more input signals. The SLD output signal may specify one or more wavelengths of light (or spectral regions) to be delivered or filtered. Typically, one spectral region of the SLD used as part or all of the output signal of the controller is the region of spectral light that is involved in ipRGC activation or attenuation (e.g., 440 nm to 520 nm (520 nm to 480 nm:blue-green; 480 nm to 450 nm:blue; 440 nm to 430 nm:indigo)). Other spectral regions may form part of the SLD including but not limited to 700-640 nm (red), 640-625 nm (orange-red), 625-615 nm (orange), 615-600 nm (amber), 600-585 nm (yellow), 585-555 nm (yellow-green), 555-520 nm (green), 440-430 nm (indigo), or 430-395 nm (violet). As indicated herein, the SLD often has one or more time dependencies which may include duration (continuous length the SLD is to be delivered), timing (e.g., during 24 hour period or recurring 24 hour periods or wake cycle), or length of treatment regimen (e.g., over one day, 1 week, 1 month one year, etc.). The controller may format the SLD in a variety of manners taking into account information from the input signals. A preliminary SLD may be determined by the controller which undergoes further refinement until to selected or desired lighting environment is determined. For example, the preliminary SLD can specify spectral regions or wavelengths that are to be delivered or filtered (e.g., for ipRGC activation or attenuation). Other wavelengths of light may be determined to not have substantial impact on the treatment. The preliminary SLD (or SLD) may also take into account the one or more illuminants (or filters) of the systems and methods described herein. Once the relevant regions of the SLD are determined, in some aspects, the intensity of relevant regions (see below ILD) of the SLD may be determined. The SLD may then be modified to further optimize one or more various parameters of light including, but not limited to, CRI, color temperature, color balancing, luminosity, luminance, etc.

Intensity of Light Delivered (ILD) (122): The ILD can be one of one or more of the output signals derived from the controller based on the one or more input signals. The ILD typically involves a specification of intensity of light of one or more wavelengths of light over the ipRGC range (e.g., 440 nm to 520 nm (520 nm to 480 nm:blue-green; 480 nm to 450 nm:blue; 440 nm to 430 nm:indigo)) where the intensity is determined to yield a desired effect on ipRGC activity (e.g., activation, attenuation, or maintenance), ultimately modulating autonomic function in the individual in relation to a target disease condition or disorder. The ILD output signal may also include intensity of light at wavelengths or region outside of the ipRGC range 700-640 nm (red), 640-625 nm (orange-red), 625-615 nm (orange), 615-600 nm (amber), 600-585 nm (yellow), 585-555 nm (yellow-green), 555-520 nm (green), 440-430 nm (indigo), or 430-395 nm (violet).

Duration of Light Exposure (DLD) (124): The DLD can be one of the one or more of the output signals derived from the controller based on the one or more input signals. The DLD represents the amount of time a particular spectrum and intensity of light is to be delivered to an individual by the systems and methods described herein. Thus, output signal related to DLD can be any amount of time that an individual is to be exposed to particular lighting conditions to achieve the desired effect. Typically, the DLD is specified as an amount of time (e.g., 30 seconds, 1 minute, 2, minute, 3 minutes, 5 minutes, 10 minutes, 15 minutes, 30 minutes, 1 hour, 2 hours, 3 hours, 4 hours, 6 hours, 8 hours, 10 hours, 12 hours, or 14 hours or more) within the 24 hour day. The DLD, in some aspects, is often linked to timing during the day when the light is to be delivered or modulated (TLE). The DLD typically depends on or is related to the target disease, disorder or condition and other input factors to achieve the desired effect.

Timing of Light Exposure (TLE) (126): The TLE can be one of the one or more of the output signals derived from the controller based on the one or more input signals. The TLE represents the time of day or time of wake cycle a particular spectrum and intensity of light is to be delivered to an individual by the systems and methods described herein. Thus, output signal related to TLE can be any amount time of day or wake cycle that an individual is to be exposed to particular lighting conditions to achieve the desired effect. Typically, the TLE is specified as a time of day (e.g., any time of day) within the 24 hour day or wake cycle. The TLE, in some aspects, is often linked to the amount of time the light that is to be delivered or modulated (DLD). The TLE typically depends on or is related to the target disease, disorder or condition and other input factors to achieve the desired effect.

Length of Light Treatment Regimen (LLR) (128): The LLR can be one of the one or more of the output signals derived from the controller based on the one or more input signals. The LLR can be one of the one or more of the output signals derived from the controller based on the one or more input signals. The LLR represents the period of time that the systems and methods described herein are to be used by the individual to achieve the desired effect. Thus, output signal related to TLE can be any period of time of time that an individual is to use the systems or methods described herein. Typically, the LLR is specified as a period of time (e.g., 1 day, 2 days, 3 days, 4 days, 5 days, 6 days, 1 week, 2 weeks, 3 weeks, 4 weeks, 1 month, 2 months, 3 months, 4 months, 5, months, 6 months, 9 months, 12 months, 1 year, 2 years or more) within the 24 hour day. The LLR typically depends on or is related to the target disease, disorder or condition and other input factors to achieve the desired effect.

As shown in FIG. 3, one, two, three, four or five (or more) input signals corresponding to HLE (100), ALC (102), ATI (104), DSI (106), and DDC (108) are communicated to a controller (150) which has the capability to process the input signals and generate one, two, three, four or five (or more) output signals corresponding to (120), (122), (124), (126), and (128). The output one, two, three, four or five (or more) signals are typically communicated to a driver (160). The input signals can be directly communicated to the controller or may be communicated to the controller via one or more intermediate devices. The specific input signal can be communicated once in some situations or can be communicated multiple times or continuously. For example, HLE or DDC may be communicated once, although in some situations this information may be communicated more than one time. But generally, HLE and DDC are static parameters. In some configurations an individual e.g., a user or a healthcare worker, physician, nurse or other third party may input HLE or DDC information into the controller. Such input can be remote or directly. Typically, but not always, input signals like ATI and DSI are signals that are delivered multiple times or continuously to the controller. These signals often are obtained from the one or more sensors of the systems and methods described herein. Signals like ATI or DSI can be transmitted wireless to the controller or may be transmitted via a physical connection like a wire or cable. Alternatively, a user or other individual may enter such inputs like ATI or DSI directly or indirectly to the controller by manually entering values corresponding to ATI or DSI via e.g., selecting values using a touch screen interface or keyboard. For example, in some aspects, the controller may be a computer or smartphone or the like and DSI or ATI e.g., obtained from one or more sensors are input into the systems via a keyboard or touch screen. Alternatively, the input signals can be input into a secondary device and then transmitted to the controller from secondary device.

The one or more output signals (120, 122, 124, 126, and 128) are typically transmitted to a driver (160). Again, in some output signals are more likely, although not necessarily, transmitted to the driver (160) one time whereas other output signals may be transmitted multiple times or continuously. It should be noted that the controller (150) and driver (160) are typically denoted as two separate units throughout this application—this representation is functional and not necessarily physical. For example, the controller and driver may be configured as one physical unit or they may be physically distinct. Additional, throughout this application the controller and driver are each often represented as a single unit but it is intended that there may be multiple controllers or drivers. Thus, each driver when indicated as a single unit in a figure or passage is intended to represent 1 driver or multiple drivers (e.g., 1, 2, 3, 4, 5, 6, 7, 8, 9, or 10 or more). Similarly, each controller, when indicated as a single unit in a figure or passage is intended to represent 1 controller or multiple controllers (e.g., 1, 2, 3, 4, 5, 6, 7, 8, 9, or 10 or more). Furthermore, although the inputs signals and output signals are represented as single entities they are intended to refer to single entities and multiple entities (e.g., 1, 2, 3, 4, 5, 6, 7, 8, 9, or 10 or more).

It should be noted that some figures and passages depict or convey direct connections to or from the devices of the system and method, they are intended to encompass other configuration which involve intermediate or indirect connections. For example, input signals may be communicated to a third-party device or site and then transmitted back to the controller. Thus, it is contemplated that the third-party site may be a computer or similar device that is monitored and/or manipulated by a nurse, physician, health care worker or other individual and this information may be transmitted then to the controller. Likewise, similar situations for output signals are also contemplated herein.

One feature of the controller is to determine a desired or selected lighting environment. The desired or selected lighting environment can be determined in a variety of manners. At a basic level, this can involve determining an ipRGC activation level in the individual. As a non-limiting example, in some aspects, the ipRGC activation level is estimated or calculated from input signals corresponding one or more of ATI or DSI. The ATI or DSI provide a measure of autonomic function. In the context of the desired outcome of modulating, treating or preventing a pathological or physiological state, the controller may specify that an alteration in the ipRGC activation level of the individual (subject or user) is desirable. This may be represented symbolically as ΔipRGC. The ΔipRGC can be used as a basis for altering the lighting environment by determining the light in the ipRGC range that is needed in addition to the ALC to bring about this ΔipRGC or determining the light in the ipRGC range that is needed to be removed (e.g., by optical filtering) to bring about this ΔipRGC.

In some aspects, the desired or selected lighting environment involves an optimization of the light. As described herein, various light optimization protocols may be employed such as disclosed in U.S. Pat. Nos. 7,663,739, 8,009,278, and 7,626,693 or elsewhere. Thus, in some aspects, the optimization protocol involves determining a lighting environment sufficient to provide a desired level of ipRGC activation or attenuation and then optimizing other optical indices. For example, the controller may determine that a certain amount of light is to be emitted from a particular illuminant and once this amount is determined, optimizations of other lighting parameters can be made to e.g., to optimize CRI, color balancing, color temperature, luminance, luminosity or other characteristics. Other light optimization protocols are known by the skilled artisan and may likewise be employed in this manner or any other manner.

In some embodiments, the controller of the system and method described herein may specify other outputs including, but not limited to, behavior modifications (timing of sleep, diet (food or drink) and diet habits (time of day of eating or drinking)) or modification in exposure to external stimuli (e.g., noise, sound, stressors, relaxors, etc.).

In some embodiment, the controller instead of specifying to a driver a lighting change it may specify the user to manually control illuminants in the environment or use a particular optical filter to control the lighting (e.g., eyeglasses or other ophthalmologic products configured to modulate ipRGC activity).

The driver as used in the systems and methods described herein provides the power to an illuminant or illuminants. The driver is operably linked (wireless or wired) to the controller from which it receives information about whether to power a particular illuminant or illuminants. Some drivers may be specific to the type of illuminant that it is driving. For example LED illuminants need specific power supplies depending on the exact type of LED. Furthermore, LEDs have power needs that may vary depending on the temperature of the LED and thus have specific drivers that account for these variations. Dimmable LED drivers may be used with the systems and methods described herein. Examples of LED drivers include, but are not limited to, Xitanium from Philips or Lightech™ from GE lighting. In one aspect, the driver is one or more constant current drivers that delivers in the range of 1 W 350 mA-48 W 1050 mA. In one aspect, the driver is one or more phase control dimmable drivers that operate at either 110 V or 220 V and can deliver 18 W, 26 W and 36 W; 350 mA, 500 mA, 700 mA, and 1050 mA. In aspect the driver is a 1-10 dimmable driver that delivers 18 W-36 W, 350 mA-700 mA and 48 W 1050 mA.

According to the systems and methods provided herein, information is transmitted between the sensors, drivers, controllers and/or illuminants. In some aspects, the information is transmitted wirelessly. Systems and methods described herein can use various types of hardware and software to accomplish this information transfer. For example, a ZigBee system or a system employing Zigbee architecture can be used in the systems and methods described herein.

Illuminants (or optical filters) are configured to be capable of modulation of spectral irradiance to an individual at luminosities and spectral regions that regulate autonomic tone (e.g., modulate ipRGC activity). Thus, the illuminants are capable of altering the luminance (e.g., exposed to a person) in the range of light of about 440 nm to 520 nm (e.g., 520-480 nm (blue-green), 480-450 nm (blue), or 450-4440 nm (indigo)), and optionally altering the luminance exposed to a person in the range of light of 300 nm to 440 nm or 520 nm to 700 nm. In some aspects, the one or more illuminants for the range of light in the 440 nm to 520 nm range are configured to expose an individual to irradiances of at least 10⁴, 10⁵, 10⁶, 10⁷, 10⁸, 10⁹ or 10¹⁰ photons/cm²/sec over a time period sufficient to modulate ipRGC activity. In some aspects, the one or more illuminants for the range of light in the 440 nm to 520 nm range are configured to expose an individual to irradiances 10⁶ to 10¹⁶ photons/cm²/sec over a time period sufficient to modulate ipRGC activity. In some aspects, the one or more optical filters for the range of light in the 440 nm to 520 nm range (or 460 nm to 500 nm) are configured to expose an individual to irradiances of less than 10⁴, 10⁵, 10⁶, 10⁷, or 10⁸ photons/cm²/sec over a time period sufficient to modulate ipRGC activity. In some aspects, the one or more optical filters for the range of light in the 440 nm to 520 nm range (or 460 nm to 500 nm) are configured to expose an individual to irradiances of 10⁰ to 10⁸ photons/cm²/sec (or 10¹ to 10⁶ photons/cm²/sec) over a time period sufficient to modulate ipRGC activity.

The optional one or more illuminants (or one or more optical filters) that modulate light in the range of 300 nm to 440 nm or 520 nm to 700 nm are configured to modulate or optimize one or more optical indices such as color rendering, color temperature, color balancing, luminance, or luminosity; or they are configured to modulate the autonomic tone effect of the illuminants producing light in the range of 440 nm to 520 nm. The optional illuminant(s) are capable in modulating luminance in one or more ranges selected from 700-640 nm (red), 640-625 nm (orange-red), 625-615 nm (orange), 615-600 nm (amber), 600-585 nm (yellow), 585-555 nm (yellow-green), 555-520 nm (green), 440-430 nm (indigo) or 430-395 nm (violet). The optional illuminant may be used to alter the quality of light e.g., color rendering, luminance, luminosity, color balancing, color temperature, and/or function to modulate ipRGC signal through the rod/cone system of the eye.

Illuminants as described herein may involve filters placed on windows to control external lighting or with other artificial or natural illuminants. The illuminant specified by this invention may involve a spectral array of illuminants of different colors which are adjusted as necessary to achieve the specifications provided by this invention.

The illuminant specified by this invention may be incandescent, fluorescent, a light emitting diode (LED), an array of light emitting diodes, a laser diode (LD), an array of laser diodes, or any other laser device or array thereof, any diffuse or specular reflection device coupled with any source of light, or a combination of any other illuminants specified herein or elsewhere.

The illuminant may be a broad area illuminant, full room illuminant, partial room illuminant, or spot or localized illuminant. In a particular embodiment, the illuminant is a heliostat.

The illuminant may be a display device such as a CRT, LCD, plasma, or any display mechanism involving other illuminants specified under this invention. An illustrative, but not limiting example might be for instance a computer display screen.

The illuminant specified under this invention might be that projected onto a typical reflection display screen as seen in movie theatres and classrooms. In this case the illuminant is both that which imparts light onto the display screen or use of the reflective properties of the screen itself, in which the screen itself is defined as an illuminant.

The systems and methods described herein may involve ophthalmic or non-ophthalmic eyewear specified in combination with use of an illuminant or in particular lighting environments.

The illuminant in some aspects and systems described herein may comprise a RF-embedded light bulb or light bulb systems, such as that of Insteon. Other examples of illuminant systems for use herein include, but not limited to, may involve the use Greenwave Reality NXP's JenNet-IP protocol or the ZigBee Light Link to control the color and light level of LED light bulbs.

Examples of LED or LED systems useful in the systems and methods of the invention include, but are not limited to, Philips LumiLEDS (e.g., Luxeon (LUXEON A, LUXEON Flash, LUXEON H, LUXEON K, LUXEON M, LUXEON Mid-Power (LUXEON Mid-Power 3535 or LUXEON Mid-Power 5630), LUXEON R, LUXEON Rebel LUXEON Rebel White, LUXEON Rebel Color, LUXEON Rebel PLUS, LUXEON S, LUXEON T, LUXEON Z, or SuperFlux) Intellilight, EssentialColor, or IntelliPower Dynamic LED lighting), Osram LED downlight (e.g., SOLERIQ E FAMILY, Duris E, Duris P, Golden Dragon Plus, Oslon SSL series, Oslon Square, Osram OSTAR lighting plus,), GE LEDs (Lumination LED Luminaires—e.g., EP, EL, ET, BL or IP series). See e.g., U.S. Pat. No. 6,590,235, U.S. Pat. No. 5,779,924, U.S. Pat. No. 6,717,353, U.S. Pat. No. 6,547,249, and U.S. Pat. No. 6,274,924.

In some embodiments, one component (e.g., illuminant or optical filter) of the system provides an ipRGC response suitable for modulating autonomic function, autonomic tone, sympathetic activity, parasympathetic activity, sympathetic reactivity, or parasympathetic reactivity. In some aspects, the range (or notch) for ipRGC response control can be a total of 2, 5, 10, 15, 20, 25, 30, 35, 40, 45, 50, 55, 60, 65, 70, 75 or more nm wide (can be one contiguous band or two, three, four, or five or more noncontiguous bands (e.g., 2 notches, 3, notches, 4 notches or 5 notches or more)), centered at 460, 465, 470, 475, 480, 485, 490, 495, 500, 505, 510, or 515 nm. In one aspect, the illuminant is one or more illuminants that deliver 10%, 20%, 30%, 40%, 50%, 60%, 70%, 80%, 90% or 95% or more of the light constituting the ipRGC response range. In another aspect, the illuminant is filtered with one or more optical filters that blocks 10%, 20%, 30%, 40%, 50%, 60%, 70%, 80%, 90% or 95% or more of the light constituting the ipRGC response range. In some aspects, the one or more illuminants selectively deliver desired wavelengths for modulating ipRGC response and does not substantially deliver light below 450 nm, 445 nm, 440 nm, 435 nm or 430 nm (e.g., delivers or transmits less than 20%, 15%, 10% or 5% of light in this range). In some aspects, the one or more illuminants selectively deliver desired wavelengths for modulating ipRGC response and does not substantially deliver above 520 nm, 530 nm, 540 nm, 550 nm or 575 nm (e.g., delivers or transmits less than 20%, 15%, 10% or 5% of light in this range). In some aspects, the optical filter selectively blocks desired wavelengths for modulating ipRGC response (e.g., transmits less than 20%, 15%, 10% or 5% of light in this range) and blocks light transmission below 440 nm, 430 nm, 420 nm, 410 nm or 400 nm (e.g., transmits less than 20%, 15%, 10% or 5% of light in this range). In some aspects, the optical filter selectively blocks desired wavelengths for modulating ipRGC response (e.g., delivers or transmits less than 20%, 15%, 10% or 5% of light in this range) and does not substantially block light transmission above 520 nm, 530 nm, 540 nm, 550 nm or 575 nm (e.g., blocks less than 20%, 15%, 10% or 5% of light in this range). The color rendering of the illuminant(s) or optical filter(s) can be greater than 10, 30, 50, 70, 75, 80, 85, or 90 in reference to CRI-109. The color rendering can be determined based on a selected illuminant. In some aspects, the optical filter can be configured to transmit at least some portion of light having a wavelength above about 400 nanometers (nm) and to substantially block light having a wavelength below about 400 nm. In another configuration, the optical filter is configured to transmit at least some portion of light having a wavelength below about 750 nanometers (nm) and to substantially block light having a wavelength above about 750 nm. In some aspects of this embodiment, the optical filter(s) or illuminant(s) is configured to block at least 75%, 85% or 95% of light (or not produce) having a wavelength below about 350 or 400 nanometers. In some aspects of this embodiment, the optical filter(s) or illuminant(s) is configured to block at least 75%, 85%, or 95% of light (or not produce) having a wavelength above about 700 nm, 715 nm or 730 nm. In some aspects of this embodiment, the optical filter(s) or illuminant(s) is configured to block between about 70% and about 90% light (or not produce) having a wavelength between about 500 nm and about 550 nm. In some aspects of this embodiment, the optical filter or illuminant(s) is configured to block between about 70% and about 90% light (or not produce) between about 590 nm and about 630 nm. In some aspects of this embodiment, the optical filter(s) or illuminant(s) is configured to block more than about 20%, 30%, 40%, 50%, 60%, 70%, 80%, 90% or 95% or more of at least one wavelength of light having (or not produce) a wavelength between about 440 nm and about 520 nm (or 460 nm and 500 nm). In other aspects of this embodiment, the optical filter(s) or illuminant(s) is configured to block at least 95% of at least one wavelength of light having (or not produce) a wavelength between about 440 nm and about 520 nm (or 460 and 500 nm). In some implementations, the optical filter(s) or illuminant(s) is configured to transmit between about 20% and about 30% of at least one wavelength of light having a wavelength between about 520 nm and about 540 mm. In some aspects, the optical filter or illuminant is configured to block between about 85% and about 95% of at least one wavelength of light (or not produce) having a wavelength between about 560 nm and about 580 nm. In other aspects, the optical filter or illuminant is configured to transmit between about 15% and about 25% of at least one wavelength of light having a wavelength between about 600 nm and about 620 nm. In some aspects of this embodiment, the optical filter or illuminant(s) is configured to: (a) block between about 70% and 90% or more of light (or not produce) having a wavelength between about 460 nm and 500 nm and (b) block less than less than about 20% of light (or not produce) having a wavelength above about 520 nm. The optical filter or illuminant(s), in some aspects, can be any optical filter that filters light that is exposed to an individual or delivers light to an individual. In some aspects, the optical filter is an eyewear lens or lenses, part of an eyewear system, flip-up, goggle, window, windshield, television screen or computer screen.

Embodiments of the optical filters disclosed herein can comprise any suitable materials that yield the optimized filter. Non-limiting examples of suitable materials include organic, inorganic, polymeric or a composite (e.g., combination) thereof. Once the filters specifications are optimized as described herein (e.g., for an ipRGC response, color temperature, color balancing, color rendering, and/or other optical indices), the skilled artisan is capable of selecting materials for the filter and manufacturing the filter. For example, a filter can comprise a substrate including glass (e.g., borosilicate glass), polycarbonate, plastic, polymer, etc. An example of a suitable substrate (at least for certain filter layer materials is 8511 Glass manufactured by Corning Corporation, U.S.A. In some embodiments, the substrate (e.g., 8511 Glass) is configured to transmit light having a wavelength above about 400 nanometers (nm) and to substantially block light having a wavelength below about 400 nm. By way of further examples, filter layers coupled to the substrate can comprise Niobium (Nb), such as, for example, Niobium Pentoxide (Nb₂O₅); and/or comprise Silicon (Si), such as, for example, Silicon Oxide (SiO₂). Other substrates can include tantalum (e.g., tantalum oxides).

In some embodiments, an illuminant is provided herein for use in treating, preventing or modulating alertness; arousal; vigilance; heart rate; pupil reflex; hormone levels (cortisol or melatonin levels); galvanic skin response; other physiological states; migraine headache; pain; cardiovascular conditions; diabetes; cancer; bowel disorders; psychiatric conditions or disorders; mood disorders; depression; anxiety; chronic fatigue syndrome; post-traumatic stress disorder (PTSD); attention deficit hyperactivity disorder (ADHD); attention deficit disorder (ADD); circadian cycle entrainment or maintenance; sleep disorders; obesity; insulin resistance; hypercholesterolemia; heart failure; peripheral vascular disease; vagotonic symptoms; malfunction or dysfunction of body systems/organs connected to the autonomic nervous system like the blood vessels, stomach, intestine, liver, kidneys, bladder, genitals, lungs, pupils and muscles of the eye, heart, and sweat, salivary, or digestive glands; constipation; fullness of stomach; erectile dysfunction; urinary retention urinary incontinence; orthostatic hypotension; sleep disorders; obstructive sleep apnea syndrome; congenital central alveolar hypoventilation syndrome; REM sleep behavior disorder; narcolepsy; vasovagal syncope; circadian variation in autonomic tone; circadian variation in autonomic tone; side-effects of pharmaceuticals that alter autonomic tone or function, bipolar mania; schizophrenia; multiple sclerosis (MS), brain injury; spinal cord injury; obesity; metabolic syndrome; rheumatoid arthritis; allergy; inflammatory bowel disease; asthma; Th1 immune response; Th2 immune response; tumor angiogenesis, tumor invasion, tumor cell-signaling, fibromyalgia, epilepsy, type-2 diabetes, type-1 diabetes; autonomic neuropathy; hypervigilance; or newborn (or infant)infant colic or discomfort. In one aspect, of these embodiments, the illuminant provides light (at one or more wavelengths) in the range of 440 nm to 520 nm. In one aspect, of these embodiments, the illuminant provides light (at one or more wavelengths) in the range of 520-480 nm (blue-green), 480-450 nm (blue), or 450-440 nm (indigo)). In one aspect of these embodiments, the illuminant also provides light in the range of light of 300 nm to 440 nm or 520 nm to 700 nm. In some aspects, the illuminants that provides light in the 440 nm to 520 nm range is configured to expose an individual to irradiances of at least 10⁴, 10⁵, 10⁶, 10⁷, 10⁸, 10⁹ or 10¹° photons/cm²/sec over a time period sufficient to modulate ipRGC activity. In some aspects, the illuminants for the range of light in the 440 nm to 520 nm range are configured to expose an individual to irradiances 10⁶ to 10¹⁶ photons/cm²/sec over a time period sufficient to modulate ipRGC activity. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments an optical filter is provided herein for use in treating, preventing, or modulating alertness; arousal; vigilance; heart rate; pupil reflex; hormone levels (cortisol or melatonin levels); galvanic skin response; other physiological states; migraine headache; pain; cardiovascular conditions; diabetes; cancer; bowel disorders; psychiatric conditions or disorders; mood disorders; depression; anxiety; chronic fatigue syndrome; post-traumatic stress disorder (PTSD); attention deficit hyperactivity disorder (ADHD); attention deficit disorder (ADD); circadian cycle entrainment or maintenance; sleep disorders; obesity; insulin resistance; hypercholesterolemia; heart failure; peripheral vascular disease; vagotonic symptoms; malfunction or dysfunction of body systems/organs connected to the autonomic nervous system like the blood vessels, stomach, intestine, liver, kidneys, bladder, genitals, lungs, pupils and muscles of the eye, heart, and sweat, salivary, or digestive glands; constipation; fullness of stomach; erectile dysfunction; urinary retention urinary incontinence; orthostatic hypotension; sleep disorders; obstructive sleep apnea syndrome; congenital central alveolar hypoventilation syndrome; REM sleep behavior disorder; narcolepsy; vasovagal syncope; circadian variation in autonomic tone; circadian variation in autonomic tone; side-effects of pharmaceuticals that alter autonomic tone or function, bipolar mania; schizophrenia; multiple sclerosis (MS), brain injury; spinal cord injury; obesity; metabolic syndrome; rheumatoid arthritis; allergy; inflammatory bowel disease; asthma; Th1 immune response; Th2 immune response; tumor angiogenesis, tumor invasion, tumor cell-signaling, fibromyalgia, epilepsy, type-2 diabetes, type-1 diabetes; autonomic neuropathy; hypervigilance; or newborn (or infant)infant colic or discomfort. In one aspect, of these embodiments, the optical filter filters light (at one or more wavelengths) in the range of 440 nm to 520 nm. In one aspect, of these embodiments, the optical filter filters light (at one or more wavelengths) in the range of 520-480 nm (blue-green), 480-450 nm (blue), or 450-440 nm (indigo)). In one aspect of these embodiments, the optical filer also filters light in the range of light of 300 nm to 440 nm or 520 nm to 700 nm. In some aspects, the optical filter for the range of light in the 440 nm to 520 nm range (or 460 nm to 500 nm) are configured to expose an individual to irradiances of less than 10⁴, 10⁵, 10⁶, 10⁷, or 10⁸ photons/cm²/sec over a time period sufficient to modulate ipRGC activity. In some aspects, the one or more optical filters for the range of light in the 440 nm to 520 nm range (or 460 nm to 500 nm) are configured to expose an individual to irradiances of 10⁰ to 10⁸ photons/cm²/sec (or 10¹ to 10⁶ photons/cm²/sec) over a time period sufficient to modulate ipRGC activity. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

The system(s) and method(s) described herein are configured to modulate a physiological or pathological state in an individual (or a group of individuals). Generally, the physiological or pathological state is modulated via generating a spectral irradiance profile that modulates ipRGC activity. The desired effect on the physiological or pathological state is modulated through ipRGC activity based on the one or more indices of autonomic tone that are sensed by the sensors as described herein. Thus, the systems and methods are useful for modulating or correcting one or more of the four basic autonomic imbalances: high parasympathetic activity, low parasympathetic activity, high sympathetic activity, and low sympathetic activity thereby treating, preventing or modulating a particular physiological or pathological state. Through correction or modulation of these imbalances a number of acute and chronic states can be treated, prevented, or modulated including diseases, disorders or conditions. These diseases, disorders or conditions include psychiatric (or psychological), neurological, metabolic, cardiovascular, and oncological. For example, decreased levels of parasympathetic tone or increased levels of sympathetic tone have been linked to obesity, insulin resistance, diabetes, hypertension, hypercholesterolemia, depression, anxiety, heart failure, and peripheral vascular disease. Alterations in parasympathetic and sympathetic activity are known to be involved in various cancers, neurological diseases, psychological or psychiatric diseases and so on.

The physiological or pathological state can be a specific target or a group of targets. For example, a specific target can be alertness, arousal, vigilance, heart rate, pupil reflex, hormone levels (e.g., cortisol or melatonin levels), galvanic skin response, or other physiological states. The specific targets can be the one or more indices of autonomic tone that are measured or monitored by the sensors used in the system(s) and method(s) described herein. Such specific targets are referred to as proximal states. Additionally, the specific targets can be related pathological (referred to as distal states) states like pain, cardiovascular conditions (e.g., hypertension), diseases like diabetes and cancer, bowel disorders, metabolic syndrome, psychiatric conditions like mood disorders, depression, anxiety, chronic fatigue syndrome, migraine headache, post-traumatic stress disorder (PTSD), attention deficit hyperactivity disorder (ADHD), attention deficit disorder (ADD), circadian cycle entrainment or maintenance, sleep disorders, obesity, insulin resistance, hypercholesterolemia, heart failure, and peripheral vascular disease. Other relevant diseases include diseases having or related to vagotonic symptoms. Additional diseases related to malfunction or dysfunction of body systems/organs connected to the autonomic nervous system like the blood vessels, stomach, intestine, liver, kidneys, bladder, genitals, lungs, pupils and muscles of the eye, heart, and sweat, salivary, and digestive glands. Symptoms of autonomic dysfunction may also be targeted with the systems and methods described herein and include, but are not limited to constipation, fullness of stomach, erectile dysfunction, urinary retention urinary incontinence, or orthostatic hypotension. Sleep disorders include, but are not limited to, obstructive sleep apnea syndrome (Bradley and Floras 2009), congenital central alveolar hypoventilation syndrome (Ogren et al 2010), REM sleep behavior disorder (Lanfranchi et al 2007), and narcolepsy (Plazzi et al 2011) are associated with autonomic dysfunction and can be treated, prevented or modulated with the systems and methods described herein. The pathological state target in one aspect of the systems and methods described herein is vasovagal syncope. In some aspects, the methods and systems are useful for altering circadian variation in autonomic tone, altering altered circadian variation in autonomic tone, arousal, vigilance, modulation of side-effects of pharmaceuticals that alter autonomic tone or function, bipolar mania, schizophrenia, MS, migraine, brain injury, or spinal cord injury. In a particular aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments, the system and method described herein can be used to treating, preventing or modulating an immune related condition. In one aspect, the immune related condition is rheumatoid arthritis, allergy, inflammatory bowel disease, asthma, modulating Th1 immune response, or modulating Th2 immune response. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments, the system and method provided herein can be used to treat, prevent or modulate tumor angiogenesis; tumor invasion; tumor cell-signaling; fibromyalgia; epilepsy; type-2 diabetes; or obesity. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments, the system and method provided herein can be used to treat, prevent or modulate tumor angiogenesis; tumor invasion; tumor cell-signaling; fibromyalgia; epilepsy; type-2 diabetes; or obesity. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments, the system and method provided herein can be used to treat, prevent or modulate hypervigilance.

In some embodiments, the system and method provided herein can be used to treat, prevent or modulate newborn (or infant) colic or discomfort. In one preferred aspect, the system and method disclosed herein is useful for modulating autonomic tone in a subject having any of the disease, disorders or condition described in this paragraph.

In some embodiments, the system and method provided herein can be used to treat, prevent or modulate an autonomic neuropathy (diabetic, cardiac, or cardiovascular) or acute autonomic neuropathy. In one preferred aspect, the system and method disclosed herein is useful for modulating hypervigilance in a subject having any of the disease, disorders or condition described in this paragraph. In one preferred aspect, the system and method disclosed herein is useful for modulating autonomic tone in a subject having any of the disease, disorders or condition described in this paragraph.

In some embodiments, the systems and methods described herein involve the use of sensors for detecting or measuring ATI. The ATI are used to drive lights that modulate ipRGC activity in an individual. Other disease specific indices (DSI) may also be used to refine the level of ipRGC activity modulation and improve outcome. DSI may be determined by sensors or reporting or measurement of symptoms of diseases.

The system(s) and method(s) can be used to prevent or delay the onset of symptoms or diseases described herein. Therefor the system(s) and method(s) can be used to prevent or delay the onset of physiological or pathological states. For example, the system(s) and method(s) can involve identifying an individual or group of individuals in need of modulation of autonomic tone and utilizing the system(s) and method(s) to prevent or delay the onset of the physiological or pathological state or a symptom thereof.

In some aspects, the method(s) involves providing or obtaining a diagnosis for an individual that indicates or suggests the individual can benefit from the use of or treatment with the method(s) and system(s) described herein.

Additionally, the system(s) and methods(s) described herein can be used in conjunction with pharmacological approaches to modulating autonomic function or treatments for specific conditions. For example, beta-adrenergic blocker (e.g., non-selective beta-blockers alprenolol, bucindolol, carteolol, carvedilol, labetalol, nadolol, oxprenolol, penbutolol, pindolol, propranolol, sotalol, or timolol; or beta-selective blockers acebutolol, atenolol, betaxolol, bisoprolol, celiprolol, esmolol, metoprolol, or nebivolol), adrenergic agonist (e.g., clonidine, guanfacine, guanabenz, guanethidine, xylazine, tizanidine, methyldopa, or fadolmidine), or an anti-cholinergic agent (e.g., anti-muscarinic agents like benztropine, ipratropium, oxitropium, tiotropium, glycopyrrolate, oxybutynin, tolterodine, chlorphenamine, diphenhydramine, dimenhydrinate; or anti-nicotinic agents like bupropion, hexamethonium, tubocurarine, dextromethorphan, mecamylamine, or doxacurium).

The data from the sensors used in the systems and methods described herein will often be communicated to physicians (or other interested parties) in a transmittable form that can be communicated or transmitted thereto. Such a form can vary and can be tangible or intangible. The data can be embodied in descriptive statements, diagrams, photographs, charts, images or any other visual forms. For example, graphs showing sensor levels or variation or light characteristics can be used by an appropriate party to monitor or alter treatment. The statements and visual forms can be recorded on a tangible medium such as papers, computer readable media such as floppy disks, compact disks, etc., or on an intangible medium, e.g., an electronic medium in the form of email or website on internet or intranet. In addition, data can also be recorded in a sound form and transmitted through any suitable medium, e.g., analog or digital cable lines, fiber optic cables, etc., via telephone, facsimile, wireless mobile phone, internet phone and the like. Thus, the information and data can be produced anywhere in the world and transmitted to a different location.

The systems and methods described herein can be implemented using hardware, software or a combination thereof in one or more computer systems or other processing systems capable of effectuating the systems and methods described herein.

The computer-based analysis function can be implemented in any suitable language and/or browsers including the use of cloud-based technology. Non-limiting examples of such languages include C language, object-oriented high-level programming languages, (e.g., Visual Basic, SmallTalk, C++) and the like. Applications for use in the systems and methods described herein can be written to suit commonly employed environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis (and control) functions of the systems and methods described herein can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out analysis of data obtained from the one or more sensors and optionally one or one variables such as time of day, information regarding the light environment, etc. These computer program instructions may be loaded onto a computer or other programmable apparatus to produce a machine, such that the instructions which execute on the computer or other programmable apparatus implementing the functions or steps described above to effectuate the operation of the systems and methods described herein. Computer program instructions can also be stored in a computer-readable memory or medium that can direct a computer or other programmable apparatus to function in a particular manner, such that the instructions stored in the computer-readable memory or medium produce an article of manufacture including instructions for implementing the analysis or specifying control functions. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the systems and methods described herein.

One non-limiting example of a program function for use herein comprises (a) obtaining or inputting data from the one or more sensors described herein, (b) determining an autonomic function value based on, at least in-part, the data from the one or more sensors, (c) determining a desired autonomic function value, (d) determining a lighting environment that is likely to produce the desired autonomic function value or cause the autonomic function value to become more similar to the desired autonomic value, (e) driving the lightning environment to the lighting environment determined in step (d). These steps can be performed on one module of the system or can be performed on different modules of the systems described herein. In some aspects, the computer program operates iteratively, and the effect of changes in the lighting environment on the data obtained from the one or more sensors is used to alter the lighting environment to bring about additional changes in the lightning environment to provide a lightning environment that is likely to produce the desired autonomic function value. For example, if, after one iteration, the autonomic function value is unaltered, altered away from the desired autonomic function value, or is not moving towards the desired autonomic function value, the program can specify a change in the lighting environment that may correct for these deficiencies.

In a particular embodiment, a computer system may include at least one input module for entering patient data into the computer system manually, directly from the one or more sensors or both. The computer system may include at least one output module for indicating a lighting environment that has an increased likelihood of producing the desired autonomic function value or driving the autonomic function value in the direction of the desired autonomic function value. Thus, the computer system may include at least one memory module in communication with the at least one input module and the at least one output module.

Memory modules are known to the ordinary and may include, e.g., a removable storage drive (including but not limited to, a magnetic tape drive, a floppy disk drive, a VCD drive, a DVD drive, an optical disk drive, etc.) The removable storage drive may be compatible with a removable storage unit such that it can read from and/or write to the removable storage unit. Removable storage unit may include a computer usable storage medium having stored therein computer-readable program codes or instructions and/or computer readable data. For example, removable storage unit may store patient data. Examples of removable storage unit are well known in the art, including, but not limited to, floppy disks, magnetic tapes, optical disks, and the like. The at least one memory module may also include a hard disk drive which can be used to store computer readable program codes or instructions, and/or computer readable data.

In addition, the at least one memory module may further include an interface and a removable storage unit that is compatible with interface such that software, computer readable codes or instructions can be transferred from the removable storage unit into computer system. Examples of interface and removable storage unit pairs include, e.g., removable memory chips (e.g., EPROMs or PROMs) and sockets associated therewith, program cartridges and cartridge interface, and the like. Computer system may also include a secondary memory module, such as random access memory (RAM).

Computer system may include at least one processor module. It should be understood that the at least one processor module may consist of any number of devices. The at least one processor module may include a data processing device, such as a microprocessor or microcontroller or a central processing unit. The at least one processor module may include another logic device such as a DMA (Direct Memory Access) processor, an integrated communication processor device, a custom VLSI (Very Large Scale Integration) device or an ASIC (Application Specific Integrated Circuit) device. In addition, the at least one processor module may include any other type of analog or digital circuitry that is designed to perform the processing functions described herein.

In computer modules used in the systems and methods described herein, the module typically has at least one memory module, the at least one processor module, and optionally a secondary memory module are all operably linked together through communication infrastructure, which may be a communications bus, system board, cross-bar, etc.). Through the communication infrastructure, computer program codes or instructions or computer readable data can be transferred and exchanged. Input interface may operably connect the at least one input module to the communication infrastructure. Likewise, output interface may operably connect the at least one output module to the communication infrastructure.

The at least one input module may include, for example, a keyboard, mouse, touch screen, scanner, a link (e.g., for receiving from other devices e.g., USB port, Ethernet port, WiFi, Bluetooth, etc.) and other input devices known in the art. The at least one output module may include, for example, a display screen, such as a computer monitor, TV monitor, or the touch screen of the at least one input module; a printer; audio speakers; a link (e.g., for transferring data to other devices e.g., USB port, Ethernet port, WiFi, Bluetooth, etc.); or other output devices known in the art. Computer system may also include, modems, communication ports, network cards such as Ethernet cards, and newly developed devices for accessing intranets or the internet.

In one specific aspect, a method of the invention can be described in reference to FIG. 3. 200 represents the step of detecting one or more ATI or DSI. The one or more ATI or DTI are communicated to controller 210, via a communication link 205, which determines a lighting environment to achieve an ipRGC activity that is capable of modulating the one or more ATI or DSI. The lighting environment is communicated to a driver 220, via communication link 215, that outputs power, via link 225, sufficient to drive one or more illuminants 230 that provide light sufficient to modulate ipRGC activity.

The systems and methods described herein may be used in any setting. In particular, the systems and methods described herein can be configured to be used in hospitals, residential setting, work-place setting, geriatric facilities, elder care homes, exercise facilities, command centers, and the such.

### EXAMPLES

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Effect of Lighting Environment on Autonomic Tone, Autonomic Function Reactivity, Sympathetic Reactivity, and Parasympathetic Reactivity

Heart rate variability (HRV) is taken as a measure of autonomic (tone) and is measured according to the Task force of European Society of Cardiology and North American Society of Pacing and Electrophysiology (1996). Subject lie down comfortably and electrocardiogram (EKG) recordings are made under standardized conditions after the patient has rested for 10 minutes. HRV signal is analyzed by an HRV analysis system.

The HRV data is analyzed by time domain and frequency domain. Time domain analysis determines parasympathetic activity while frequency domain analysis determines both sympathetic and parasympathetic activity. Poincare analysis is a nonlinear method that can be used to interpret HRV including parasympathetic activity, sympathetic activity and vagosympathetic balance.

Tests for autonomic function reactivity are conducted with a standard battery of tests, according to methodology reported in the literature. Sympathetic reactivity is assessed with the cold pressor test, hand-grip test, and head-up tilt test. Parasympathetic reactivity is assessed with a deep-breathing test, cold face test, and the head-up tilt test.

Autonomic reactivity tests: a polygraph and a handgrip dynamometer, a light and small handgrip dynamometer is used for the isometric exercise test. A mercury sphygmomanometer is used to measure the blood pressure.

The hand grip test, cold pressor test, and blood pressure changes due to the head up tilt test provide a measure of the sympathetic reactivity, whereas the deep breathing test, cold face test and the difference between minimum and maximum heart rate due to the head up tilt test provide a measure of the parasympathetic reactivity.

Groups of subjects (e.g., 20 in each arm) are subjected to this battery of tests in the presence and absence of light sufficient to activate ipRGC response. The time of day is controlled for and studies are conducted to determine the effect of ipRGC response at relevant times of day (e.g., upon waking, middle of day (e.g., 10 AM, 11 AM, 12 AM), evening (e.g., 6 PM, 7 PM, 8 PM, 9 PM, 10 PM—and middle of night (e.g., 12 PM, 1 AM, 2 AM, 3 AM) and in relevant populations (e.g., subjects having diseases, disorders or conditions associated with altered autonomic function).

These studies are designed and controlled to minimize the effect of variables aside from the ipRGC activation variable on the results.

Results are analyzed using appropriate statistical analysis (e.g., one-tailed or two-tailed ANOVA). It is anticipated that significant differences will be observed in the populations that are exposed to light that provides ipRGC activation as opposed to light that does not provide ipRGC activation.

### Example 2

### Brain Imaging of Areas Related to Autonomic Tone

The studies described in this example use functional magnetic resonance (fMRI) brain imaging to identify local hemodynamic changes that reflect local neural activity.

Possible stimuli for eliciting a sympathetic response include pain, fear, anticipation, anxiety, concentration or memory, cold pressor, Stroop test, breathing tests, and maximal hand grip. Examples of parasympathetic stimuli are the Valsalva maneuver and paced breathing. The responses to stimuli (e.g., one or more ATI like heart rate, heart rate variability, blood pressure, galvanic skin response, papillary response) is monitored to compare the data obtained from fMRI.

Using fMRI and electrocardiographic data (or other ATI) are obtained simultaneously, in subjects exposed to light sufficient to stimulate ipRGC activity and light not sufficient to stimulate ipRGC activity, brain correlates of autonomic function are identified in subjects performing MRI-compatible task or exposed to MRI compatible stimuli. Statistical analysis of data from measurable brain regions having autonomic involvement or are correlated with autonomic involvement (e.g., such as higher brain regions (limbic or cortical) like the parabrachial nucleus, amygdala, hypothalamus, periaquaductal gray area, posterior insula, prefrontal cortex,) are expected to show differences in the ipRGC activated group as opposed to the group not having ipRGC activation.

fMRI studies on stressor-evoked blood pressure reactivity can be used to assess the effect of ipRGC activation in corticolimbic areas, including the cingulate cortex, insula, amygdala, and cortical and subcortical areas that are involved in hemodynamic and metabolic support for stress-related behavioral responses.

Results are analyzed using appropriate statistical analysis (e.g., one-tailed ANOVA or two-tailed ANOVA). It is anticipated that significant differences will be observed in the populations that are exposed to light that provides ipRGC activation as opposed to light that does not provide ipRGC activation.

These studies are designed and controlled to minimize the effect of variables aside from the ipRGC activation variable on the results.

### Example 3

### Biomarker Measurements—Salivary Cortisol and Alpha-Amylase and Blood Melatonin Levels

Initial clinical testing is conducted on a recruited subject population of 20 men and women ages 18-40 for each arm of the study. Subjects are not on prescription medication. Subjects are placed in a room having a lighting system that is capable of modulating ipRGC activity in the subject by providing sufficient amounts of light in the ipRGC activity range (e.g., 440 nm to 520 nm, or 460 nm to 500 nm). Biomarker levels (salivary cortisol, salivary alpha-amylase or melatonin) can be measured using any appropriate method. For example, Eagle Biosciences provides a Salivary Cortisol Elisa kit; AssayMax Human Amylase ELISA Kit available at Assaypro is useful for detecting salivary alpha-amylase; melatonin can be determined with a melatonin ELISA (e.g., from IBL international). Biomarker levels can be measured in subjects at specified time increments. Subjects are in 4 groups: Group 1 contains subjects that are be monitored over a 4 hour period at ten minute intervals for biomarker levels—these patients are exposed to light that activates ipRGC response for the 4 hour period; Group 2 contains subjects that are monitored over a 4 hour period at ten minute intervals for biomarker levels and are subjected to light that is not expected to activate ipRGC response; Group 3 contains subjects that are exposed to a stimulus that alters biomarker levels (e.g., Trier Social Stress Test (TSST), electric stimulation, or pharmacological approaches) that are monitored over a 4 hour period at ten minute intervals for biomarker levels—these patients are exposed to light that activates ipRGC response for the 4 hour period; group 4 contains subjects that are exposed to a stimulus that alters biomarker levels (e.g., Trier Social Stress Test (TSST), electric stimulation, or pharmacological approaches) that are monitored over a 4 hour period at ten minute intervals for biomarker levels—these patients are exposed to light that is not expected to activate ipRGC response for the 4 hour period.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences in biomarker levels will be observed in the populations that are exposed to light that provides ipRGC activation as opposed to light that does not provide ipRGC activation.

### Example 4

### Galvanic Skin Responses

The effects of ipRGC activation on galvanic skin response can be determined using a galvanic skin response sensor and a light system that can modulate ipRGC activation, and appropriate pools of subjects that are exposed or not exposed to stimulus that alter galvanic skin response. Galvanic skin response sensors are available from a number of sources, one example of which is the NeuLog Galvanic Skin Response (GSR) Logger Sensor.

Initial clinical testing are conducted on a recruited subject population of 20 men and women ages 18-40 for each arm of the study. Subjects are not on prescription medication. Subjects are placed in a room having a lighting system that is capable of modulating ipRGC activity in the subject by providing sufficient amounts of light in the ipRGC activity range (e.g., 440 nm to 520 nm, or 460 nm to 500 nm). Galvanic skin response can be measured using any appropriate method. Galvanic skin response can be measured in subjects at specified time increments including continuous monitoring. Subjects are in 4 groups: Group 1 contains subjects that are monitored over a 4 hour period for galvanic skin response—these patients are exposed to light that activates ipRGC response for the 4 hour period; Group 2 contains subjects that are monitored over a 4 hour period for galvanic skin response and are subjected to light that is not expected to activate ipRGC response; Group 3 contains subjects that are exposed to a stimulus that alters galvanic skin response (e.g., using the International Affective Picture System (IAPS), physical stimulation (e.g., remote pin prick), or pharmacological approaches) that are monitored over a 4 hour period for galvanic skin response—these patients are exposed to light that activates ipRGC response for the 4 hour period; group 4 contains subjects that are exposed to a stimulus that alters galvanic skin response (e.g., using the International Affective Picture System (IAPS), physical stimulation (e.g., remote pin prick), or pharmacological approaches) that are monitored over a 4 hour period for galvanic skin response—these patients are exposed to light that is not expected to activate ipRGC response for the 4 hour period.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences in galvanic skin response will be observed in the populations that are exposed to light that provides ipRGC activation as opposed to light that does not provide ipRGC activation.

### Example 5

### Heart Function Measures

The effects of ipRGC activation on heart function measures can be determined using a galvanic skin response sensor and a light system that can modulate ipRGC activation, and appropriate pools of subjects that are exposed or not exposed to stimulus that alter heart function. Heart function sensors are available from a number of sources, one example of which is the an EKG device like the GE MAC 1200 EKG Machine or alternatively devices such as Polar FT80 fitness heart rate monitor.

Initial clinical testing are conducted on a recruited subject population of 20 men and women ages 18-40 for each arm of the study. Subjects are not on prescription medication. Subjects are placed in a room having a lighting system that is capable of modulating ipRGC activity in the subject by providing sufficient amounts of light in the ipRGC activity range (e.g., 440 nm to 520 nm, or 460 nm to 500 nm). Heart function is measured using any appropriate method. Heart function is measured in subjects at specified time increments including continuous monitoring. Subjects are in 4 groups: Group 1 contains subjects that are monitored over a 4 hour period for heart function—these patients are exposed to light that activates ipRGC response for the 4 hour period; Group 2 contains subjects that are monitored over a 4 hour period for heart function and are subjected to light that is not expected to activate ipRGC response; Group 3 contains subjects that are exposed to a stimulus that alters heart function (e.g., using the International Affective Picture System (IAPS), physical stimulation (e.g., exercise), or pharmacological approaches) that are monitored over a 4 hour period for heart function—these patients are exposed to light that activates ipRGC response for the 4 hour period; group 4 contains subjects that are exposed to a stimulus that alters heart function (e.g., using the International Affective Picture System (IAPS), physical stimulation (e.g., exercise), or pharmacological approaches) that are monitored over a 4 hour period for heart function—these patients are exposed to light that is not expected to activate ipRGC response for the 4 hour period.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences in heart function will be observed in the populations that are exposed to light that provides ipRGC activation as opposed to light that does not provide ipRGC activation.

### Example 6

### Blood Glucose Levels

The effects of ipRGC activation on blood glucose can be determined using a blood glucose meter and a light system that can modulate ipRGC activation, and appropriate pools of subjects that are exposed or not exposed to stimulus that alter blood glucose. Blood glucose meters are available from a number of sources, ACCU-CHEK® Nano Meter or the OneTouch line of products.

Initial clinical testing are conducted on a recruited subject population of 20 men and women ages 18-40 for each arm of the study. Subjects are not on prescription medication. Subjects are placed in a room having a lighting system that is capable of modulating ipRGC activity in the subject by providing sufficient amounts of light in the ipRGC activity range (e.g., 440 nm to 520 nm, or 460 nm to 500 nm). Blood glucose can be measured using any appropriate method. Blood glucose can be measured in subjects at specified time increments e.g., 15 minute intervals. Subjects are in 4 groups: Group 1 contains subjects that are monitored over a 4 hour period for blood glucose—these patients are exposed to light that activates ipRGC response for the 4 hour period; Group 2 contains subjects that will be monitored that are monitored over a 4 hour period for blood glucose and are subjected to light that is not expected to activate ipRGC response; Group 3 contains subjects that are exposed to a stimulus that alters blood glucose (e.g., treatment with glucose after fasting or after a meal, or exercise, or emotional stimulation that alters blood glucose) that are monitored over a 4 hour period for blood glucose—these patients are exposed to light that activates ipRGC response for the 4 hour period; group 4 contains subjects that are exposed to a stimulus that alters blood glucose (e.g., treatment with glucose after fasting or after a meal, or exercise, or emotional stimulation that alters blood glucose) that are monitored over a 4 hour period blood glucose—these patients are exposed to light that is not expected to activate ipRGC response for the 4 hour period.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences in blood glucose will be observed in the populations that are exposed to light that provides ipRGC activation as opposed to light that does not provide ipRGC activation.

### Example 7

### Vigilance Trial

The psychomotor vigilance test (PVT) can be used to monitor the effect of light in the ipRGC range on psychomotor vigilance (See e.g., Basner M, Dinges D F Sleep (2011) 34(5):581-91)

Initial clinical testing are conducted on a recruited subject population of 20 men and women ages 18-40 for each arm of the study. Subjects are not on prescription medication. Subjects are placed in a room having a lighting system that is capable of modulating ipRGC activity in the subject by providing (or not providing) sufficient amounts of light in the ipRGC activity range (e.g., 440 nm to 520 nm, or 460 nm to 500 nm). The PVT can be administered to the subjects in each group as follows. Subjects are in 4 groups: Group 1 contains subjects that are not fatigued—these patients are exposed to light that activates ipRGC response and given the PVT (during the day); Group 2 contains subjects that are monitored over a 4 hour period that are fatigued and are subjected to light that is not expected to activate ipRGC response (during the day); Group 3 contains subjects that are not fatigued that are—these patients are exposed to light that activates ipRGC response (at night) and then given the PVT; Group 4 contains subjects that are fatigued—these patients are exposed to light that does not activate ipRGC response (at night) and are then given the PVT. The fatigued subjects are chosen as such due to sleep loss.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences in psychomotor will be observed in the populations that are exposed to light that provides ipRGC activation as opposed to light that does not provide ipRGC activation.

### Example 8

### Arousal or Attention Trial

Subjects are divided into 4 groups ((1) normal subjects exposed to light that does not activate ipRGC response; (2) normal subjects exposed to light that activates ipRGC response; (3) arousal deficient subjects exposed to light that does not activate ipRGC response and (4) arousal deficient subjects exposed to light that activates ipRGC response) and are to complete tasks that engage the anterior and posterior attention networks (continuous performance task, go/no-go task, and cued target detection task). During the performance of the three attentional tasks, tonic and phasic arousal, are measured. Cortical measures of arousal include e.g., frequency band power, theta/beta ratios over frontal and parietal cortices, and P300 amplitude and latency over parietal cortices. Peripheral measures of arousal include e.g., skin conductance responses, heart rate and heart rate variance. Participants rate their perceived mental effort during each of the three attentional tasks. These studies are designed and controlled to minimize the effect of variables aside from the ipRGC activation variable on the results.

A similar trial can be conducted using measures of attention e.g., test of variables of attention (see the world wide web tovatest.com).

Results are analyzed using appropriate statistical analysis (e.g., one-tailed ANOVA or two-tailed ANOVA). It is anticipated that significant differences will be observed in the populations that are exposed to light that provides ipRGC activation as opposed to light that does not provide ipRGC activation.

### Example 9

### Modulation of PTSD

The Structured Clinical Interview for DSM-IV (First, Spitzer, Gibbon, & Williams, 1996) is used to assess diagnostic criteria for PTSD before including participants in the study. The Impact of Event Scale Revised (IES-R) is used to evaluate subjects during the course of the study (Weiss, D.S., & Marmar, C. R. (1997). The Impact of Event Scale-Revised. In J.P. Wilson, & T. M. Keane (Eds.), Assessing Psychological Trauma and PTSD: A Practitioner's Handbook (pp. 399-411). New York: Guilford Press.). Subjects are divided into two basic groups: (1) treatment with light for a period of time sufficient to stimulate ipRGC activity and (2) treatment with light for a period of time not sufficient to stimulate ipRGC activity (light similar to that exposed to group (1) by and illuminant or filtered to not have light that stimulates ipRGC activity). The physiological effects of light stimulation are measured as described herein (e.g., ECG and ICG (impedance cardiogram)) during the light treatment period. Subjects in both treatment groups are exposed to cues (internal representations or external stimuli (e.g., auditory or visual)) that alter one or more ATI after onset of treatment with or without light that modulates ipRGC activity. The course of the study is over 4 weeks with one session per week. The IES-R is evaluated 1-week prior to the commencement of session 1 and 1 week after termination of session 4. It is expected that differences between the group 1 and group 2 subjects will be noted by statistical analysis of the physiological parameters (e.g., one or more ATI) and/or the IES-R questionnaire.

In a second study 60 subjects (30 in each arm) having combat related PTSD are identified and tested for startle response under conditions of ipRGC activation and lack of ipRGC activation. Electromyogram, skin conductance, and heart function are measured in both groups to estimate autonomic function.

These studies are designed and controlled to minimize the effect of variables aside from the ipRGC activation variable on the results.

Results are analyzed using appropriate statistical analysis (e.g., one-tailed ANOVA or two-tailed ANOVA).

### Example 10

### Hypervigilance Trial

Initial clinical testing is conducted on a recruited subject population of 20 men and women ages 18-40 for each arm of the study. Subjects are not on prescription medication. Subjects are placed in a room having a lighting system that is capable of modulating ipRGC activity in the subject by providing (or not providing) sufficient amounts of light in the ipRGC activity range (e.g., 440 nm to 520 nm, or 460 nm to 500 nm). Subjects are monitored for ATI and measurements of hypervigilance. Subjects are in 4 groups: Group 1 contains subjects that are hypervigilant—these patients are exposed to light that activates ipRGC response and exposed to stimuli that increase hypervigilant activity; Group 2 contains subjects that are hypervigilant and exposed to stimuli that does not increase hypervigilant activity and are subjected to light that is not expected to activate ipRGC response (during the day); Group 3 contains subjects that are not hypervigilant and are exposed to stimuli that increase hypervigilant activity—these patients are exposed to light that activates ipRGC response; Group 4 contains subjects that are not hypervigilant exposed to stimuli that does not increase hypervigilant activity—these patients are exposed to light that does not activate ipRGC response.

These studies are designed and controlled to minimize the effect of variables aside from the ipRGC activation variable on the results.

Results are analyzed using appropriate statistical analysis (e.g., one-tailed ANOVA or two-tailed ANOVA). It is anticipated that significant differences hypervigilant activity (and/or ATI) will be observed in the populations that are exposed to light that provides ipRGC activation as opposed to light that does not provide ipRGC activation.

### Example 11

### Infant Trial

Initial clinical testing are conducted on a subject population of 20 infants or newborns for each arm of the study. Subjects are not on prescription medication. Subjects will be monitored with one or more sensors typically used in monitoring newborn or infant s in a hospital setting. The infant or newborn room has a lighting system that is capable of modulating ipRGC activity in the subject by providing (or not providing) sufficient amounts of light in the ipRGC activity range (e.g., 440 nm to 520 nm, or 460 nm to 500 nm). Subjects are monitored for one or more ATI or DSI and also measures of comfort or discomfort are made such as crying, movement, symptoms of colic and the such. Subjects are in 4 groups: Group 1 subjects are exposed to light that activates ipRGC response—the ambient light has ipRGC activating characteristics (subjects not selected for any condition e.g., a general population); Group 2 contains subjects that are exposed to light that is not expected to activate ipRGC response (by filtering of the light in the ipRGC range) (subjects not selected for any condition e.g., a general population); Group 3 subjects is a selected population are exposed to light that activates ipRGC response—the ambient light has ipRGC activating characteristics (subjects are selected for a discomfort condition e.g., excessive crying, excessive movement, or symptoms of colic); Group 4 contains are subjected to light that is not expected to activate ipRGC response (by filtering of the light in the ipRGC range) ((subjects are selected for a discomfort condition e.g., excessive crying, excessive movement, or symptoms of colic).

These studies are designed and controlled to minimize the effect of variables aside from the ipRGC activation variable on the results.

Results are analyzed using appropriate statistical analysis (e.g., one-tailed or two-tailed ANOVA). It is anticipated that significant differences in discomfort level and/or ATI and DSI will be observed in the populations that are exposed to light that provides ipRGC activation as opposed to light that does not provide ipRGC activation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference and as far as they are consistent with the disclosure herein. The mere mentioning of the publications and patent applications does not necessarily constitute an admission that they are prior art to the instant application.

All of the compositions, methods and systems disclosed and claimed herein can be made and executed without undue experimentation in light of the present disclosure. While the compositions and methods of this invention have been described in terms of particular embodiments, it will be apparent to those of skill in the art that variations may be applied to the compositions, methods and systems and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A system comprising (a) one or more sensors operably linked to (b) a controller operably linked to (c) a driver operably linked to (d) one or more illuminants or one or more optical filters capable of modulating light.

2. The system of claim 1 wherein the one or more illuminants or one or more optical filters provide light or filter light in a range of the spectrum that modulates ipRGC activity.

3. The system of claim 1 the one or more illuminants or one or more optical filters provide light or filter light in a range 440 nm to 520 nm or 460 nm to 500 nm.

4. The system of claim 1 wherein the controller comprises a CPU and computer readable medium having computer readable instructions for determining a desired or selected lighting environment using data from the one or more sensors.

5. The system of claim 1 wherein the one or more illuminants are selected from incandescent, fluorescent, a light emitting diode (LED), an OLED, an array of light emitting diodes or OLEDs, a laser diode (LD), an array of laser diodes, or any other laser device or array thereof, any diffuse or specular reflection device coupled with any source of light, or a combination of these illuminants; a broad area illuminant, full room illuminant, partial room illuminant, or spot or localized illuminant; a display device CRT, LCD, plasma, or any display mechanism involving other illuminants; sunlight; or any combination thereof.

6. The systems of claim 1 wherein the one or more sensors sense autonomic tone, sympathetic activity, parasympathetic activity, sympathetic reactivity, or parasympathetic reactivity.

7. The systems of claim 1 wherein the one or more sensors sense one or more autonomic tone indices (ATI) or one or more disease specific indices (DSI).

8. The system of claim 1 wherein the one or more sensors sense (1) heart function (blood pressure, flow or pulse), (2) gastrointestinal activity (movements or secretions), (3) body temperature, (4) bronchial dilation, (5) blood glucose levels, (6) metabolism, (7) micturition or defecation, (8) pupillary light or accommodation reflexes, (9) glandular secretions, (10) heart rate variability (RF or LF component), (11) cortisol level, (12) alpha-amylase level, or (13) melatonin level.

9. A method for modulating autonomic tone said method comprising obtaining data corresponding to one or more ATI or one or more DSI, determining an autonomic tone value from the one or more ATI or one or more DSI, determining a desired autonomic tone value, determining a desired lighting environment from the autonomic tone value and the desired autonomic tone value, and providing a desired lighting environment.

10. The method of claim 9 wherein the one or more ATI or one or more DSI is a heart function parameter, galvanic skin response, a salivary biomarker or a blood biomarker.

11. The method of claim 9 wherein the one or more ATI or one or more DSI is (1) heart function (blood pressure, flow or pulse), (2) gastrointestinal activity (movements or secretions), (3) body temperature, (4) bronchial dilation, (5) blood glucose levels, (6) metabolism, (7) micturition or defecation, (8) pupillary light or accommodation reflexes, (9) glandular secretions, (10) heart rate variability (RF or LF component), (11) cortisol level, (12) alpha-amylase level, or (13) melatonin level.

12. The method of claim 9 wherein said method further comprises altering autonomic tone, sympathetic activity, parasympathetic activity, sympathetic reactivity or parasympathetic reactivity.

13. The method of claim 9 wherein said desired lightning environment alters autonomic tone, sympathetic activity, parasympathetic activity, sympathetic reactivity or parasympathetic reactivity.

14. The method of claim 9 said desired lightning environment alters one or more ATI or one or more DSI.

15. The method of claim 9 further comprising treating or preventing a disease or condition associated with altered (1) autonomic tone, sympathetic activity, parasympathetic activity, sympathetic reactivity or parasympathetic reactivity.

16. The method of claim 9 further comprising treating, preventing or modulating: alertness; arousal; vigilance; heart rate; pupil reflex; hormone levels (cortisol or melatonin levels); galvanic skin response; other physiological states; migraine headache; pain; cardiovascular conditions; diabetes; cancer; bowel disorders; psychiatric conditions or disorders; mood disorders; depression; anxiety; chronic fatigue syndrome; post-traumatic stress disorder (PTSD); attention deficit hyperactivity disorder (ADHD); attention deficit disorder (ADD); circadian cycle entrainment or maintenance; sleep disorders; obesity; insulin resistance; hypercholesterolemia; heart failure; peripheral vascular disease; vagotonic symptoms; malfunction or dysfunction of body systems/organs connected to the autonomic nervous system like the blood vessels, stomach, intestine, liver, kidneys, bladder, genitals, lungs, pupils and muscles of the eye, heart, and sweat, salivary, or digestive glands; constipation; fullness of stomach; erectile dysfunction; urinary retention urinary incontinence; orthostatic hypotension; sleep disorders; obstructive sleep apnea syndrome; congenital central alveolar hypoventilation syndrome; REM sleep behavior disorder; narcolepsy; vasovagal syncope; circadian variation in autonomic tone; circadian variation in autonomic tone; side-effects of pharmaceuticals that alter autonomic tone or function, bipolar mania; schizophrenia; multiple sclerosis (MS), brain injury; spinal cord injury; obesity; rheumatoid arthritis; allergy; inflammatory bowel disease; asthma; Th1 immune response; Th2 immune response; tumor angiogenesis, tumor invasion, tumor cell-signaling, fibromyalgia, epilepsy, type-2 diabetes, type-1 diabetes; hypervigilance; autonomic neuropathy; or newborn (or infant)infant colic or discomfort.

17. The method of claim 9 wherein said providing a desired lighting environment comprises modulating light in the range of 440 nm to 520 nm.

18. The method of claim 9 wherein said providing a desired lighting environment comprises modulating light in the range of 440 nm to 520 nm and in the range of 700-640 nm (red), 640-625 nm (orange-red), 625-615 nm (orange), 615-600 nm (amber), 600-585 nm (yellow), 585-555 nm (yellow-green), 555-520 nm (green), 440-430 nm (indigo) or 430-395 nm (violet).

19. A controller comprising (a) an input module for receiving data from one or more sensors, (b) an output module for delivering data regarding the desired lighting environment to a driver or user, (c) a computer module having computer readable code for determining a desired lighting environment based on data received from the one or more sensors.

20. The controller of claim 19 operably linked to one or more illuminants or filters that produce or filter light in the range of 440 nm to 520 nm.

21. The controller of claim 19 operably linked to one or more sensors.

22. The controller of claim 19 operably linked to a driver.

23. The controller of claim 19 operably linked to one or more illuminants or one or more filters that produce or filter light the range of 440 nm to 520 nm and in the range of 700-640 nm (red), 640-625 nm (orange-red), 625-615 nm (orange), 615-600 nm (amber), 600-585 nm (yellow), 585-555 nm (yellow-green), 555-520 nm (green), 440-430 nm (indigo) or 430-395 nm (violet).
